(19) United States
(12) Patent Application Publication
(10) Pub. No.: US 2017/0020838 A1
(43) Pub. Date: Jan. 26, 2017

(54) TREATMENT FOR RESPIRATORY DISEASE

(71) Applicant: MUCOKINETICA LIMITED, London (GB)

(72) Inventors: Roderick Lindsay HALL, Addlestone (GB); Peter John COLE, London (GB)

(73) Assignee: MUCOKINETICA LIMITED, London (GB)

(21) Appl. No.: 15/302,210

(22) PCT Filed: Apr. 7, 2015
(86) PCT No.: PCT/GB2015/051057

§ 371 Date: Oct. 6, 2016

(30) Foreign Application Priority Data
Apr. 7, 2014 (GB) 1406225.1

(51) Int. Cl. A61K 31/24; A61K 33/20; A61K 31/047; A61K 31/191; A61M 11/00; A61K 45/06; A61K 31/7072; A61K 31/4965; A61M 15/00; A61K 9/00; A61K 9/08

(52) U.S. Cl. CPC A61K 31/24; A61K 9/0078; A61K 9/0075; A61K 33/20; A61K 31/047; A61K 31/191; A61K 9/08; A61K 45/06; A61K 31/7072; A61K 31/4965; A61M 15/009; A61M 11/005; A61M 2202/064

(57) ABSTRACT

The present invention relates to a family of amidino compounds in combination with a hyperosmotic agent or a purinergic agonist for use in a method of treating respiratory disease. More specifically the invention relates to 6-amidino-2-napthyl 4-guanidinobenzoate in combination with a hyperosmotic agent or a purinergic agonist for use in a method of treating respiratory disease such as cystic fibrosis.

### CROSS-REFERENCE TO RELATED APPLICATIONS

This application is a U.S. National Phase Application of International Application No. PCT/GB2015/051057, filed Apr. 7, 2015 which claims priority to British Application No. 1406225.1, filed Apr. 7, 2014, the contents of which are incorporated herein by reference in their entireties for all purposes.

### FIELD OF INVENTION

The present invention relates to a family of amidino compounds in combination with a hyperosmotic agent or a purinergic agonist for use in a method of treating respiratory disease.

In a preferred embodiment the invention relates to 6-amidino-2-napthyl 4-guanidinobenzoate in combination with a hyperosmotic agent or a purinergic agonist for use in a method of treating respiratory disease such as cystic fibrosis.

### BACKGROUND OF THE INVENTION

In the respiratory epithelium the properties and composition of the airway surface liquid (ASL) that lines the airway surfaces are critical to effective operation of airway defense functions, especially mucociliary clearance. Absorption of sodium ions via Amiloride-sensitive sodium channels (ENaC) located at the apical surface of epithelial cells is particularly important in regulating mucociliary function in health and disease and has become a target for treating respiratory disease.

Clearance of airway secretions by cilia is a critical host defense function that helps maintain health and hygiene in the airways and lungs. The surface epithelium of the conducting airways contains ciliated cells. Mucus secretions in the airways trap dust and microorganisms that are inhaled with breathing. These secretions are then removed from the airways by the actions of the ciliated cells. The cilia beat in a coordinated manner and interact with overlying secretions to cause them to be moved up the airways towards the larynx. Here they are normally swallowed imperceptibly.

When this clearance mechanism is operating normally it helps to maintain lung heath and hygiene. Underperformance of mucociliary clearance in disease can result in a build up of infected inflamed secretions.

Mucostasis is the accumulation of secretions and defective clearance in respiratory disease. Poor clearance of secretions from the lower airways is a continuing problem and is believed to lead to chronic infection, inflammation in the airways, reduction in lung function and in some conditions destruction of the lungs and eventual respiratory failure. Lowering ENaC activity is a promising strategy to improve performance of mucociliary clearance in respiratory diseases characterised by mucostasis.

Amiloride and analogues have been evaluated in the clinic as potential inhaled therapies for treatment of respiratory diseases characterised by mucostasis, particularly cystic fibrosis. Amiloride reduces ENaC activity by direct exofacial block in the pore of the channel, but is a short-lived blocker and washing epithelial cells to remove Amiloride results in rapid recovery of ENaC activity, typically within 1 to 2 minutes. For this reason clinical results using Amiloride have been disappointing and rapid reversibility of binding to ENaC together with rapid absorption by the airway epithelium is thought to be responsible for the short duration of efficacy achieved in patients.

Inhaled hypertonic saline and inhaled dry powder mannitol are therapies that are in use clinically to treat respiratory diseases characterised by mucostasis, particularly cystic fibrosis.

Inhalation of hypertonic saline and dry powder mannitol by patients have both been shown to increase ciliary clearance of secretions in diseases where accumulation of infected inflamed secretions contributes to morbidity and mortality. Measurement of increased whole lung mucociliary clearance has proved to be a predictive biomarker in proof of concept phase II clinical trials in cystic fibrosis and non-CF bronchiectasis patients. The effects of hypertonic saline and mannitol on enhancing mucociliary clearance function may be of critical importance to delivering improvements vs clinical end points in phase III clinical studies since the efficacy of hypertonic saline or mannitol inhalation to increase mucociliary clearance of secretions in phase II trials has been predictive of efficacy vs clinical end points in phase III clinical trials.

In phase III clinical trials in cystic fibrosis patients, inhaled nebulised hypertonic saline (Elkins et al. 2006) and inhaled dry powder mannitol (Bilton et al. 2013) achieved modest benefits vs clinical end points; reductions in chronic lung infections (pulmonary exacerbations), and in some patients modest reductions in the decline of lung function (improved FEV1). In a long term inhaled hypertonic saline trial (Elkins et al. 2006), patients inhaled 4 ml nebulised 7% hypertonic saline (1.2M-NaCl; equivalent to 2400 mosM) twice daily for 48 weeks. It is generally accepted that in home nebuliser use only about 10% of the nebulised solution is respirable and deposits in a patient's airways. If the deposited hypertonic saline solution were to be evenly distributed through the airways in the approximately 10 ml of airway surface liquid and airway secretions, then the osmolarity of the airway surface liquid and secretions would be increased by approximately 100 mosM; for example from 300 mosM (isoosmolar) to 400 mosM. The therapeutic concentration window for achieving clinical benefit from osmotically active agents is likely to be narrow as demonstrated in a model of ciliary transport of mucus on a bovine trachea where maximum speed of transport was achieved with mucus and sputum samples equilibrated in hyperosmotic saline buffer solutions of 400 and 500 mosM (Wills et al. 1997) and equilibration in buffer solutions of osmotic concentrations above 500 mosM resulted in sub-maximal ciliary transport. Exposure of ciliated human nasal epithelial cells in vitro to 3% saline solution, 1000 mosM-NaCl, produced ciliostasis within a few minutes (Min et al. 2001), whereas no ciliary slowing occurred using isotonic 0.9% saline solution, 300 mosM-NaCl.

To achieve clinical benefit from osmotically active agents delivered into the lungs it is therefore important to control the amount of active agent delivered and thus to control the osmotic concentration achieved in the airway surface liquid and secretions in the lungs, and the present inventors have taken this into account during development of the present invention.

The critical molecular mechanism behind the clinical efficacy of inhaled hyperosmotic therapies may be reduction of airway ENaC activity. Rasgado et al. (2009 & 2013) demonstrated that exposure of human airway epithelial cells to hyperosmotic concentrations of saline and mannitol reduced ENaC activity, where maximum reduction of ENaC activity was achieved 15min after exposure to hypertonic saline (Rasgado et al. 2013). Hypertonic saline and dry powder mannitol are valuable treatments for respiratory disease, but there is much scope and opportunity for improvement of potential therapies that can deliver better efficacy, duration of action, therapeutic window and safety.

Down regulation of airway ENaC activity has also been achieved by Kunitz-type serine protease inhibitors, aprotinin and BAY 39-9437 (Bridges et al. 2001). The proposed mechanism of action of the Kunitz inhibitors is to inhibit endogenous proteases responsible for proteolytic activation of sodium channel proteins in the apical membrane of epithelial cells. In contrast to the short acting blocker profile of Amiloride, BAY 39-9437 is an example of a long duration down regulator of ENaC. Treatment of airway epithelial cells with BAY 39-9437 results in reduction of ENaC activity that is sustained for an extended period of time after unbound substance has been removed, by washing, from contact with the epithelial cells (Bridges et al. 2001).

6-amidino-2-napthyl 4-guanidinobenzoate, was first described in JP57053454 and is also described in U.S. Pat. No. 4,454,338 as a member of a family of related amidino compounds, which are described as having powerful antitrypsin, antiplasmin, antikallikrein, antithrombin, antigranzyme and anticompliment activity. 6-amidino-2-napthyl 4-guanidinobenzoate is also known to act as an anti-inflammatory agent (Iwaki et al. 1984) and an anticoagulant (Hitomi et al. 1985).

Muto et al. (1993) reported that 6-amidino-2-napthyl 4-guanidinobenzoate caused a change in transepithelial voltage within the cortical collecting duct of the rabbit kidney and this compound was thought to act as a weak, short acting inhibitor of the kidney sodium channel.

Rossier (2004) reviewed hormonal and serine protease regulation of the epithelial sodium channel and identified clear differences in hormonal control of the activity of ENaC in different organs and tissues. In the kidney and colon ENaC expression is under mineralocorticoid (aldosterone) control, whereas in the distal lung glucocorticoid control operates.

In WO 2008/090366, the present inventors identified for the first time that 6-amidino-2-napthyl 4-guanidinobenzoate and related compounds could be used to regulate the activity of ENaC in airway epithelial cells and could therefore be used for the treatment of respiratory diseases characterised by poor mucociliary clearance or mucostasis.

Surprisingly, the present inventors have now found that the ENaC activity inhibitory effect of 6-amidino-2-napthyl 4-guanidinobenzoate is significantly enhanced when used in combination with a hyperosmotic agent or a purinergic agonist. This and related compounds, similarly in combination with a hyperosmotic agent or a purinergic agonist, therefore have potential in the treatment of respiratory diseases characterised by mucostasis such as cystic fibrosis, bronchiectasis, chronic bronchitis, chronic obstructive pulmonary disease (COPD), rhino-sinusitis and otitis media.

The present inventors have further found that substances that block or down regulate airway ENaC activity also act to increase ciliary transport of mucus secretions in a model in the trachea. Without being bound by theory, the inventors consider that it may be this mechanism of action which permits the surprising enhancement of activity 6-amidino-2-napthyl 4-guanidinobenzoate when used in combination with a hyperosmotic agents or a purinergic agonist.

### SUMMARY OF THE INVENTION

In a first aspect the present invention is directed to an amidino compound of the general formula (I):

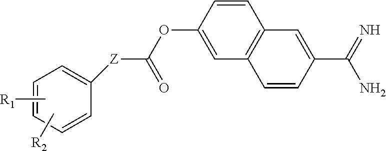

and a hyperosmotic agent or a purinergic agonist for use in a method of treating respiratory disease.

The invention is also directed to a composition comprising an amidino compound of the general formula (I):

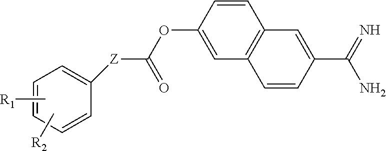

and a hyperosmotic agent or a purinergic agonist.

The composition may be for use in a method of treating respiratory disease.

The invention also encompasses an inhalation device loaded with the composition.

The invention further relates to a method of treating a respiratory disease, comprising administering a therapeutically effective amount of an amidino compound of the general formula (I):

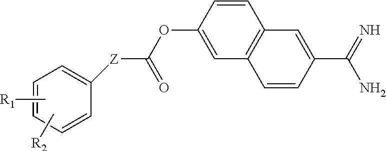

and a therapeutically effective amount of a hyperosmotic agent or a purinergic agonist.

In the formula (I), Z represents —(CH₂)a-,

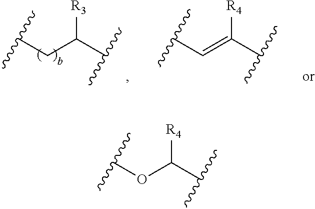

wherein a is 0, 1, 2 or 3, b is 0,1 or 2, R₃ is a straight or branched chain alkyl group of 1 to 4 carbon atoms or a cycloalkyl group of 3 to 6 carbon atoms, R₄ is a hydrogen atom or a straight or branched chain alkyl group of 1 to 4 carbon atoms.

R₁ and R₂, which may be the same or different, represent each a hydrogen atom, a straight or branched chain alkyl group of 1 to 4 carbon atoms, —O—R₅, —S—R₅, —COOR₅, —COR₆, —O—COR₇, —NHCOR₇,

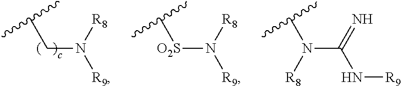

NO₂, CN, halogen, CF₃, methylenedioxy, or

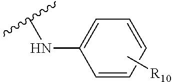

wherein c is 0, 1 or 2; R₅ is a hydrogen atom, linear or branched chain alkyl group of 1 to 4 carbon atoms, or benzyl group; R₆ is a hydrogen atom or straight or branched chain alkyl group of 1 to 4 carbon atoms; R₇ is a straight or branched chain alkyl group of 1 to 4 carbon atoms; R₈ and R₉, which may be the same or different, are each a hydrogen atom, straight or branched chain alkyl group of 1 to 4 carbon atoms, or amino radical protecting group; and R₁₀ is a hydrogen atom, methyl or CF₃. The straight or branched chain alkyl groups of 1 to 4 carbon atom include methyl, ethyl, n-propyl, isopropyl, n-butyl, isobutyl and tert-butyl.

In a second aspect the present invention is directed to an Amiloride-sensitive sodium channel (ENaC) regulator for use in a method of increasing ciliary transport of mucus secretions.

Within this aspect the Amiloride-sensitive sodium channel (ENaC) regulator may be selected from the group comprising Amiloride and other exofacial sodium channel blockers; hypertonic saline, hyperosmotic mannitol, hyperosmotic sodium gluconate, and other hyperosmotic treatments; amidino compounds and Kunitz-type serine protease inhibitors.

### BRIEF DESCRIPTION OF THE DRAWINGS

The invention will be now be described by way of exemplification only with reference to the accompanying figures, brief descriptions of which follow.

FIG. 1 A description of the equipment used to measure electrical potential difference (PD) across the pig trachea ex vivo (FIG. 1A). Pig tracheas placed on a sloping radiator heated at 37° C. A reference electrode consisting of silicone tubing filled with Signa electrode cream was attached to the outer surface of the trachea. A double lumen recording electrode (FIG. 1B) was placed inside the trachea in contact with the tracheal epithelium. The inner tubing of the recording electrode was used to perfuse onto the surface of the trachea buffer solutions and buffer solutions containing drugs and agents. The outer part of the recording electrode was also filled with Signa electrode cream. The ends of the electrodes not in contact with the trachea were placed in Ringer buffer solution. Electrical contact to calomel half-cells in 3M-KCl solution was achieved by silicone tubing bridges filled with Signa electrode cream. The calomel half-cells were connected to a high impedance digital electrometer (Agilent U1252A). Digital PD values were recorded continuously every 1 second for the duration each study and were filed onto a PC.

FIG. 2 FIG. 2A shows the amiloride-sensitive tracheal PD response in ex vivo pig trachea perfused at 1 ml/min with balanced salt solution (BSS) containing 0.145 M-NaCl, 5mM-NaHCO₃, 4.05 mM-KCl, 1.2 mM-CaCl₂, 1.2 mM-MgCl₂, 2.4 mM-K₂HPO₄, 0.4mM-KH₂PO₄, 10 mM glucose adjusted to pH7.4. PD values were recorded every 1 s for 140 min. At 20, 35, 50, 65, 80, 95, 110 and 125 min 3×10⁶M-amiloride was added to the perfusing BSS for 5 min; followed by 10 min wash out with BSS alone. FIG. 2A is the trace of the mean data from recordings from 23 pig tracheas. FIG. 2B shows the area under the curve (AUC) response (mean+SD) to amiloride perfusion for each of the eight peaks in FIG. 2A. AUC data is normalised by expressing the response for each peak as the percentage of the average response for the first four peaks.

FIG. 3 Effect of perfusion of 6-amidino-2-napthyl 4-guanidinobenzoate (Rₓ) on amiloride-sensitive tracheal PD responses in ex vivo pig trachea. Tracheas were perfused with BSS between 0 to 75 min, followed by perfusion with BSS containing 3.10⁵M 6-amidino-2-napthyl 4-guanidinobenzoate. PD values were recorded every 1 s for 140 min. At 20, 35, 50, 65, 80, 95, 110 and 125 min 3×10⁶M-amiloride was added to the perfusing BSS or BSS+Rₓ for 5min; followed by 10 min wash out with buffer. FIG. 3A is the trace of the mean data from recordings from 8 pig tracheas. FIG. 3B shows the area under the curve (AUC) response (mean+SD) to amiloride perfusion for each of the eight peaks in FIG. 3A. AUC data is normalised by expressing the response for each peak as the percentage of the average response for the first four peaks.

FIG. 4 Effect of perfusion of hyperosmotic saline (NaCl) and 6-amidino-2-napthyl 4-guanidinobenzoate (Rₓ) on amiloride-sensitive tracheal PD responses in ex vivo pig trachea. In FIG. 4A tracheas were perfused with BSS between 0 to 75 min, followed by perfusion between 75 min and 140 min with BSS containing a further 50mM-NaC1, then finally perfused between 140 min and 200 min with BSS. PD values were recorded every 1 s for 200 min. At 20, 35, 50, 65, 80, 95, 110, 125, 140, 155, 170 and 185 min 3×10⁶M-amiloride was added to the perfusing BSS or BSS+NaCl for 5min; followed by 10min wash out with buffer. FIG. 4A is the trace of the mean data from recordings from 15 pig tracheas.

In FIG. 4B tracheas were perfused with BSS between 0 to 75 min, followed by perfusion between 75 min and 140 min with BSS containing a further 50mM-NaCl and 10⁻⁵M 6-amidino-2-napthyl 4-guanidinobenzoate (Rₓ), then finally perfused between 140 min and 200 min with BSS. PD values were recorded every 1 s for 200 min. At 20, 35, 50, 65, 80, 95, 110, 125, 140, 155, 170 and 185 min 3×10⁶M-amiloride was added to the perfusing BSS or BSS+NaCl+Rₓ for 5min; followed by 10 min wash out with buffer. FIG. 4B is the trace of the mean data from recordings from 17 pig tracheas.

FIG. 5 Effect of perfusion of hyperosmotic saline (NaCl) (open columns), and hyperosmotic saline plus 6-amidino-2-napthyl 4-guanidinobenzoate (Rₓ) (filled columns) on AUC of the amiloride-sensitive tracheal PD responses (mean+95%CI) in ex vivo pig trachea in FIGS. 4A and 4B. AUC data is normalised by expressing the response for each peak as the percentage of the average response for the first four peaks.

FIG. 6 Effect of perfusion of hyperosmotic mannitol (Mannitol) and 6-amidino-2-napthyl 4-guanidinobenzoate (Rₓ) on amiloride-sensitive tracheal PD responses in ex vivo pig trachea. In FIG. 6A tracheas were perfused with BSS between 0 to 75 min, followed by perfusion between 75 min and 200 min with BSS containing 100 mM-mannitol, then finally perfused between 200 min and 260 min with BSS. PD values were recorded every 1 s for 260 min. At 20, 35, 50, 65, 80, 95, 110, 125, 140, 155, 170, 185, 200, 215, 230 and 245 min 3×10⁶M-amiloride was added to the perfusing BSS or BSS+Mannitol for 5 min; followed by 10 min wash out with buffer. FIG. 6A is the trace of the mean data from recordings from 17 pig tracheas.

In FIG. 6B tracheas were perfused with BSS between 0 to 75 min, followed by perfusion between 75 min and 200 min with BSS containing 100 mM-mannitol and 10⁵M 6-amidino-2-napthyl 4-guanidinobenzoate (Rₓ), then finally perfused between 200 min and 260 min with BSS. PD values were recorded every 1 s for 260 min. At 20, 35, 50, 65, 80, 95, 110, 125, 140, 155, 170, 185, 200, 215, 230 and 245 min 3×10⁶M-amiloride was added to the perfusing BSS or BSS+Mannitol+Rₓ for 5 min; followed by 10 min wash out with buffer. FIG. 6B is the trace of the mean data from recordings from 18 pig tracheas.

FIG. 7 Effect of perfusion of hyperosmotic mannitol (open columns), and hyperosmotic mannitol plus 6-amidino-2-napthyl 4-guanidinobenzoate (Rₓ) (filled columns) on AUC of the amiloride-sensitive tracheal PD responses (mean+95%CI) in ex vivo pig trachea in FIGS. 6A and 6B. AUC data is normalised by expressing the response for each peak as the percentage of the average response for the first four peaks.

FIG. 8 Effect of perfusion of hyperosmotic sodium gluconate on amiloride-sensitive tracheal PD responses in ex vivo pig trachea. Tracheas were perfused with BSS between 0 to 75 min, followed by perfusion with BSS containing 50 mM-sodium gluconate. PD values were recorded every is for 140 min. At 20, 35, 50, 65, 80, 95, 110 and 125 min 3×10⁶M-amiloride was added to the perfusing BSS or BSS+NaGluconate for 5 min; followed by 10 min wash out with buffer. FIG. 8 is the trace of the mean data from recordings from 6 pig tracheas.

FIG. 9 Effect of perfusion of UTP (uridine 5′-triphosphate) on amiloride-sensitive tracheal PD responses in ex vivo pig trachea. Tracheas were perfused with BSS between 0 to 75 min, followed by perfusion with BSS containing 0.1 mM-UTP. PD values were recorded every is for 140 min. At 20, 35, 50, 65, 80, 95, 110 and 125 min 3×10⁶M-amiloride was added to the perfusing BSS or BSS+UTP for 5 min; followed by 10 min wash out with buffer. FIG. 9 is the trace of the mean data from recordings from 6 pig tracheas.

FIG. 10 Effect of amiloride on ciliary transport of pig airway mucus secretions on the ex vivo pig trachea. Pig tracheas (from larynx to carina) were obtained from a local abattoir or Veterinary University.

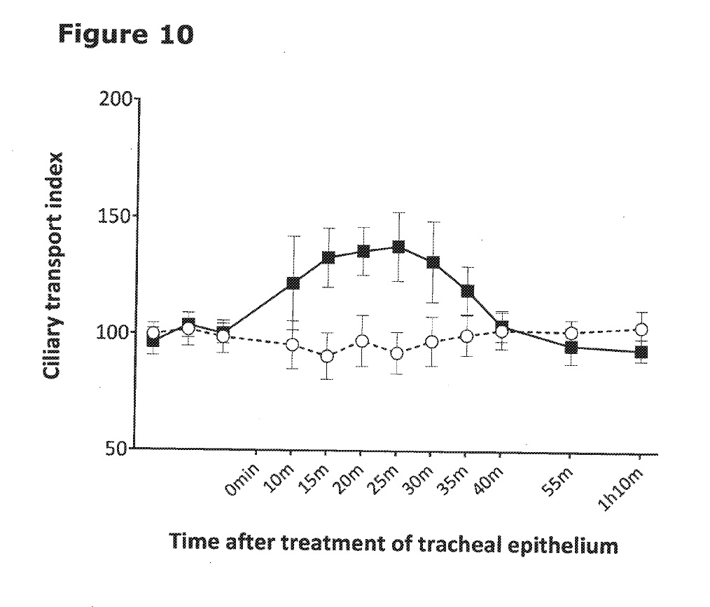

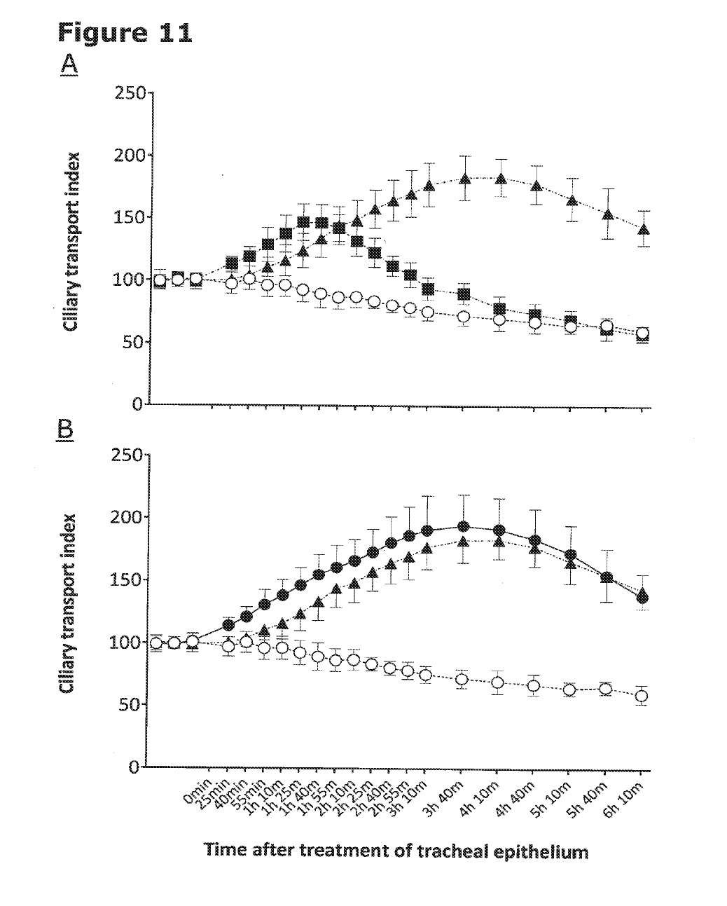

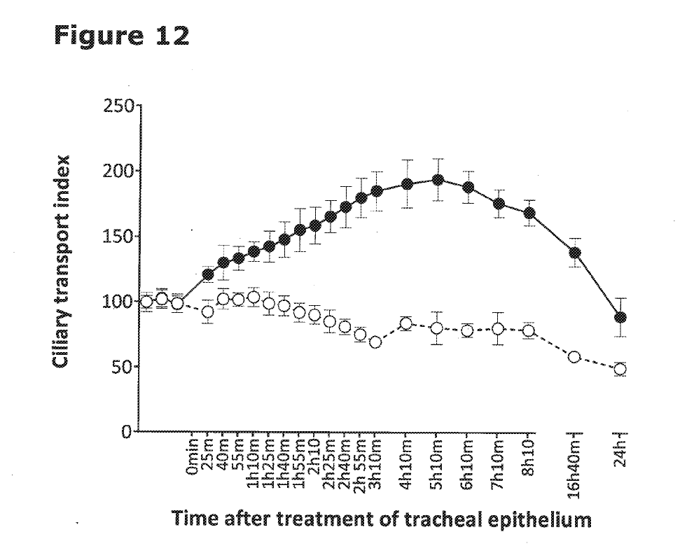

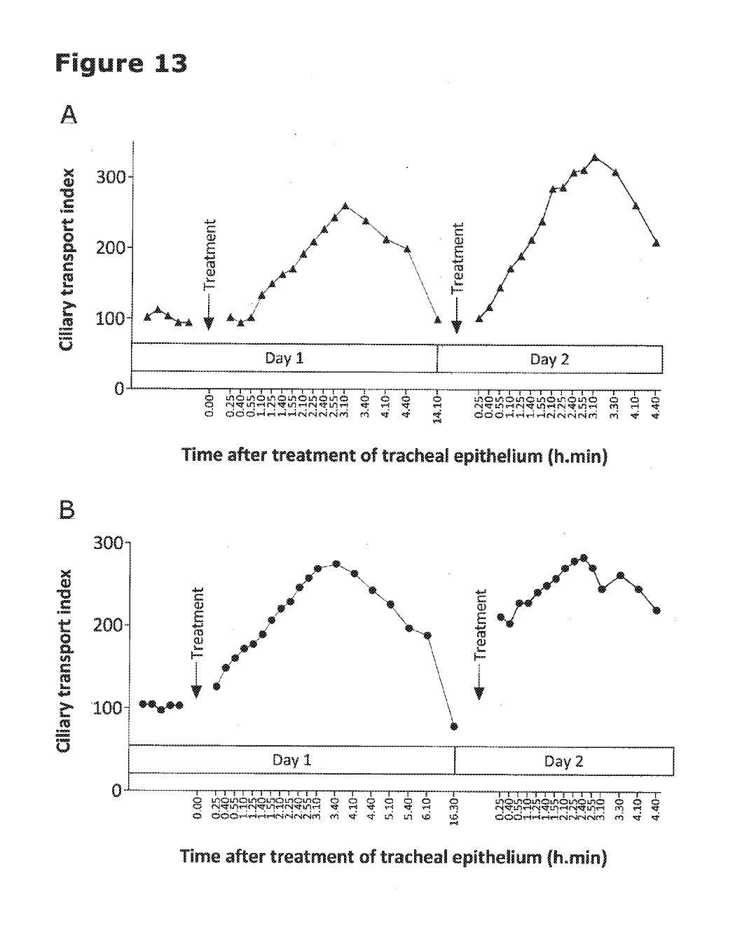

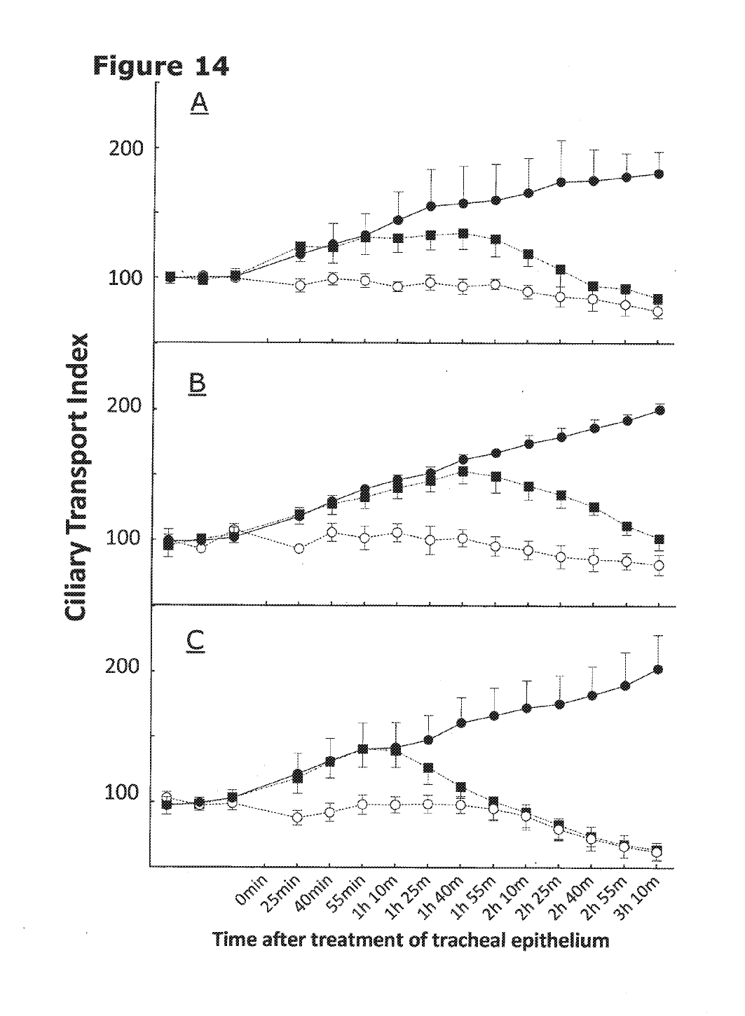

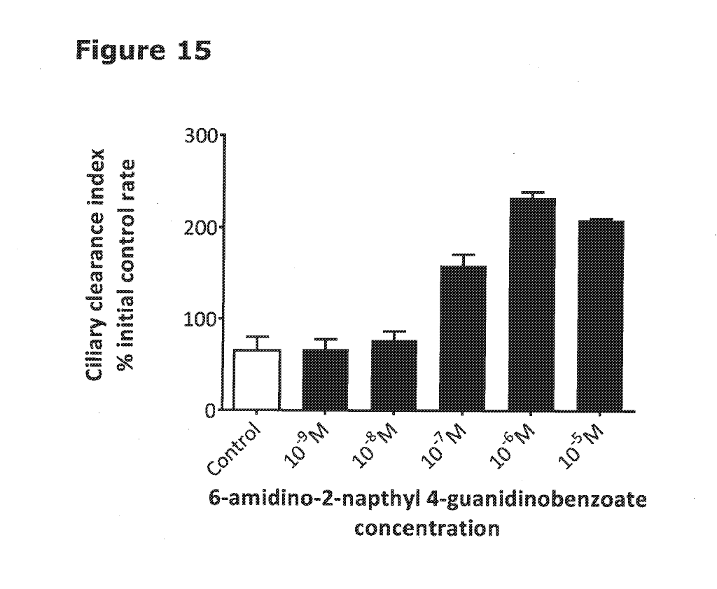

The lumen of each trachea was cleaned by gentle irrigation with BSS. Tracheas were opened longitudinally with scissors along the posterior aspect and mounted with the posterior aspect upwards in a moist air-tight box. An aliquot of pig airway mucus approximately 3 mm diameter was placed on the mucosal surface close to the distal end of each trachea. Tracheal mucus velocity (TMV mm/min) was measured by recording the distance moved, towards the laryngeal end of the trachea, in a given time. At the start of the study three measurements of TMV were made for each trachea to establish a baseline rate for the trachea. Solutions were then pipetted onto the luminal mucosal surface of tracheas for 10 min; BSS control tracheas (open circles; n=6); 10⁻⁴ M amiloride in BSS (closed squares; n=6). Excess solution was then tipped off the mucosal surface and measurement of TMV repeated. Further measurements of TMV were made at the times indicated in FIG. 10. TMV results at each time point are expressed as % of the baseline TMV, ciliary transport index (mean±SD).

FIG. 11 Study of the effects of hyperosmotic saline in BSS, 6-amidino-2-napthyl 4-guanidinobenzoate in BSS, and a combination of hyperosmotic saline and 6-amidino-2-napthyl 4-guanidinobenzoate in BSS on ciliary transport of pig airway mucus secretions on the ex vivo pig trachea by methods described in FIG. 10. Ciliary transport index (mean±SD) measured for 6 h 10 min following treatment of tracheas.

In FIG. 11A the solutions applied to the mucosal surface of tracheas were BSS control (open circles; n=10); BSS containing a further 50mM-NaCl (hyperosmotic saline solution) (closed squares; n=10); and BSS containing 10⁻⁵M 6-amidino-2-napthyl 4-guanidinobenzoate (closed triangles; n=10).

In FIG. 11B the solutions applied to the mucosal surface of tracheas were BSS control (open circles; n=10); BSS containing 10⁻⁵M 6-amidino-2-napthyl 4-guanidinobenzoate (closed triangles; n=10) and BSS containing a further 50 mM-NaCl and 10⁻⁵M 6-amidino-2-napthyl 4-guanidinobenzoate (closed circles; n=10).

FIG. 12 Study of the effects of combination of hyperosmotic saline and 6-amidino-2-napthyl 4-guanidinobenzoate in BSS on ciliary transport of pig airway mucus secretions on the ex vivo pig trachea by methods described in FIG. 10. Ciliary transport index (mean±SD) measured for 24 h following treatment of tracheas.

The solutions applied to the mucosal surface of tracheas were BSS control (open circles; n=4 to 6); and BSS containing a further 50 mM-NaCl and 10⁻⁵M 6-amidino-2-napthyl 4-guanidinobenzoate (closed circles; n=6).

FIG. 13 Study of the effects of 6-amidino-2-napthyl 4-guanidinobenzoate in BSS, and a combination of hyperosmotic saline and 6-amidino-2-napthyl 4-guanidinobenzoate in BSS on ciliary transport of pig airway mucus secretions on the ex vivo trachea of transgenic CF piglets. Transgenic CF piglets were produced as described by Klymiuk et al. 2012, and tracheas obtained at a Veterinary University. Study methods were otherwise as described in FIG. 10. Ciliary transport index (mean) was measured over two days with a second treatment period at the start of the second day.

In FIG. 13A the solutions applied to the mucosal surface of tracheas were BSS containing 10⁻⁵ M 6-amidino-2-napthyl 4-guanidinobenzoate (closed triangles; n=3).

In FIG. 13B the solutions applied to the mucosal surface of tracheas were BSS containing a further 50 mM-NaCl and 10⁻⁵ M 6-amidino-2-napthyl 4-guanidinobenzoate (closed circles; n=3).

FIG. 14 Studies of the effects of hyperosmotic mannitol in BSS, hyperosmotic sodium gluconate in BSS, and UTP in BSS, and combinations with 6-amidino-2-napthyl 4-guanidinobenzoate on ciliary transport of pig airway mucus secretions on the ex vivo pig trachea by methods described in FIG. 10. Ciliary transport index (mean±SD).

In FIG. 14A the solutions applied to the mucosal surface of tracheas were BSS control (open circles; n=7); BSS containing 100 mM-mannitol (closed squares; n=7); and BSS containing 100 mM-mannitol and 10⁻⁵M 6-amidino-2-napthyl 4-guanidinobenzoate (closed circles; n=7).

In FIG. 14B the solutions applied to the mucosal surface of tracheas were BSS control (open circles; n=5); BSS containing 50 mM-sodium gluconate (closed squares; n=6); and BSS containing 50 mM-sodium gluconate and 10⁻⁵M 6-amidino-2-napthyl 4-guanidinobenzoate (closed circles; n=6).

In FIG. 14C the solutions applied to the mucosal surface of tracheas were BSS control (open circles; n=10); BSS containing 0.1 mM-UTP (closed squares; n=10); and BSS containing 0.1mM-UTP and 10⁻⁵M 6-amidino-2-napthyl 4-guanidinobenzoate (closed circles; n=10).

FIG. 15 Dose-response study of the effects of 6-amidino-2-napthyl 4-guanidinobenzoate in BSS on ciliary transport of pig airway mucus secretions on the ex vivo pig trachea. Ciliary transport index (mean+SD; n=3 or 4) at 3 h 40 min after treatment, time of peak response.

### DETAILED DESCRIPTION OF THE INVENTION

The first aspect of the present invention relates to the inventors' surprising finding that the ENaC activity inhibitory effect of 6-amidino-2-napthyl 4-guanidinobenzoate is significantly enhanced when used in combination with a hyperosmotic agent or a purinergic agonist. The first aspect of the invention therefore relates to an amidino compound of the general formula (I):

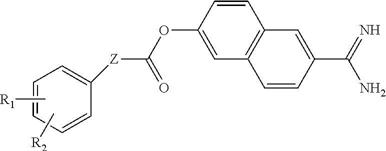

and a hyperosmotic agent or a purinergic agonist for use in a method of treating respiratory disease.

The Amidino Compound

In the formula (I), Z represents —(CH₂)a-,

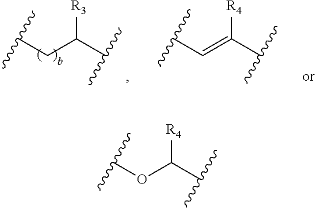

wherein a is 0, 1, 2 or 3, b is 0,1 or 2, R₃ is a straight or branched chain alkyl group of 1 to 4 carbon atoms or a cycloalkyl group of 3 to 6 carbon atoms, R₄ is a hydrogen atom or a straight or branched chain alkyl group of 1 to 4 carbon atoms.

R₁ and R₂, which may be the same or different, represent each a hydrogen atom, a straight or branched chain alkyl group of 1 to 4 carbon atoms, —O—R₅, —S—R₅, —COOR₅, —COR₆, —O—COR₇, —NHCOR₇,

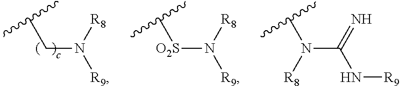

NO₂, CN, halogen, CF₃, methylenedioxy, or

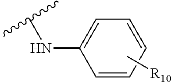

wherein c is 0, 1 or 2; R₅ is a hydrogen atom, linear or branched chain alkyl group of 1 to 4 carbon atoms, or benzyl group; R₆ is a hydrogen atom or straight or branched chain alkyl group of 1 to 4 carbon atoms; R₇ is a straight or branched chain alkyl group of 1 to 4 carbon atoms; R₈ and R₉, which may be the same or different, are each a hydrogen atom, straight or branched chain alkyl group of 1 to 4 carbon atoms, or amino radical protecting group; and R₁₀ is a hydrogen atom, methyl or CF₃. The straight or branched chain alkyl groups of 1 to 4 carbon atom include methyl, ethyl, n-propyl, isopropyl, n-butyl, isobutyl and tert-butyl.

Examples of R₁ and R₂ include hydrogen, methyl, ethyl, n-propyl, n-butyl, tert-butyl, hydroxy, methoxy, ethoxy, n-propyloxy, n-butyloxy, benzyloxy, mercapto, methylthio, ethylthio, carboxy, methoxycarbonyl, ethoxycarbonyl, butoxycarbonyl, formyl, acetyl, ethylcarbonyl, guanidino, N-methylguanidino, N-n-butylguanidino, acetoxy, propionyloxy, butyryloxy, acetamino, propionyl amino, butyrylamino, amino, dimethylamino, dibutylamino, aminomethyl, benzyloxycarbonylaminomethyl, sulfamyl, dimethylsulfamyl, nitro, cyano, fluorine, chlorine, bromine, iodine, trifluoromethyl, methylenedioxy, phenylamino, 3,4-dimethylphenylamino, and 3-trifluoromethylphenylamino.

In preferred embodiments Z represents a covalent bond.

Preferably R₁ or R₂ represent hydrogen or a straight or branched alkyl group having from 1 to 4 carbon atoms.

In particularly preferred embodiments R₁ is hydrogen and R₂ is

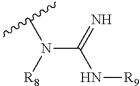

wherein R₈ and R₉ are hydrogen.

Preferably, the compound will be in the form of a pharmaceutically acceptable salt or ester.

Most preferably, the compound is 6-amidino-2-napthyl 4-guanidinobenzoate dihydrochloride or a pharmaceutically acceptable salt or ester thereof. Examples include but are not limited to 6-amidino-2-napthyl 4-guanidinobenzoate dihydrochloride and 6-amidino-2-napthyl 4-guanidinobenzoate mesylate.

Use of an Amiloride-Sensitive Sodium Channel (ENaC) Regulator to Increase Ciliary Transport of Mucus Secretions

The inventors have also surprisingly discovered that Amiloride-sensitive sodium channel (ENaC) regulators increase ciliary transport of mucus secretions. In a second aspect, the invention therefore relates to an Amiloride-sensitive sodium channel (ENaC) regulator for use in a method of increasing ciliary transport of mucus secretions.

Within this aspect, an “Amiloride-sensitive sodium channel (ENaC) regulator” is anything which functions to alter the absorption of sodium ions via Amiloride-sensitive sodium channels (ENaC). Preferably an “Amiloride-sensitive sodium channel (ENaC) regulator” will reduce or eliminate absorption of sodium ions via Amiloride-sensitive sodium channels (ENaC).

In one embodiment, the Amiloride-sensitive sodium channel (ENaC) regulator may be selected from the group comprising Amiloride and other exofacial sodium channel blockers; hypertonic saline, hyperosmotic mannitol, hyperosmotic sodium gluconate, and other hyperosmotic treatments; amidino compounds and Kunitz-type serine protease inhibitors. Further, the Amiloride-sensitive sodium channel (ENaC) regulator may be selected from the group consisting of Amiloride and other exofacial sodium channel blockers; hypertonic saline, hyperosmotic mannitol, hyperosmotic sodium gluconate, and other hyperosmotic treatments; amidino compounds and Kunitz-type serine protease inhibitors.

In certain embodiments of the second aspect of the invention, the Amiloride-sensitive sodium channel (ENaC) regulator may be an amidino compound such as an amidino compound of the general formula (I):

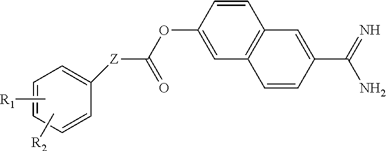

Within this embodiment all definitions of the amidino compound provided above will apply equally to the second aspect of the invention.

In one embodiment increasing ciliary transport of mucus secretions may be associated with the treatment of a respiratory disease. Herein, the respiratory disease may be selected from the group comprising cystic fibrosis, bronchiectasis, chronic bronchitis, chronic obstructive pulmonary disease (COPD), rhino-sinusitis and otitis media. Alternatively, the respiratory disease may be selected from the group consisting of cystic fibrosis, bronchiectasis, chronic bronchitis, chronic obstructive pulmonary disease (COPD), rhino-sinusitis and otitis media. Preferably, the respiratory disease is cystic fibrosis.

In one embodiment, the Amiloride-sensitive sodium channel (ENaC) regulator may be administered in combination with a hyperosmotic agent or a purinergic agonist. Within this embodiment, all definitions of the hyperosmotic agent or a purinergic agonist provided below will equally apply.

The Hyperosmotic Agent

Throughout all of the methods, uses and compositions of the first aspect of the invention, and in some embodiments of the second aspect of the invention, the amidino compound or other Amiloride-sensitive sodium channel (ENaC) regulator described above is used with a hyperosmotic agent or a purinergic agonist.

In one embodiment the amidino compound described above is used with a hyperosmotic agent.

The term hyperosmotic agent refers to any agent which is capable of increasing the osmolarity in a recipient's lungs. In one embodiment the hyperosmotic agent is capable of increasing the osmolarity of the recipient's lungs to 350 mosM-600 mosM. In other embodiments the hyperosmotic agent is capable of increasing the osmolarity of the recipient's lungs to 350 mosM-550 mosM, 350 mosM-500 mosM, 350 mosM-450 mosM, 350 mosM-400 mosM, 400 mosM-600 mosM, 400 mosM-550 mosM, 400 mosM-500 mosM or 400 mosM-450 mosM or to about 350 mosM, about 400 mosM, about 450 mosM, about 500 mosM, about 550 mosM, or about 600 mosM.

In one embodiment the increase in the osmolarity in a recipient's lung is achieved by administration of hyperosmotic solution as a nebulized aerosol formulation using a nebuliser such as a jet or ultrasonic nebulizer. In one embodiment the hyperosmotic solution may have an osmolarity of 600 mosM-3000 mosM, such as 700 mosM-2900 mosM, 800 mosM-2800 mosM, 900 mosM-2700 mosM, 1000 mosM-2600 mosM, 1000 mosM-2500 mosM, 1100 mosM-2500 mosM, 1200 mosM-2500 mosM, 1300 mosM-2500 mosM, 1400 mosM-2500 mosM, 1500 mosM-2500 mosM, 1600 mosM-2500 mosM, 1700 mosM-2500 mosM, 1800 mosM-2500 mosM, 1900 mosM-2500 mosM, 2000 mosM-2500 mosM, or about 600 mosM, about 700 mosM, about 800 mosM, about 900 mosM, about 1000 mosM, about 1100 mosM, about 1200 mosM, about 1300 mosM, about 1400 mosM, about 1500 mosM, about 1600 mosM, about 1700 mosM, about 1800 mosM, about 1900 mosM, about 2000 mosM, about 2100 mosM, about 2200 mosM, about 2300 mosM, about 2400 mosM, about 2500 mosM, about 2600 mosM, about 2700 mosM, about 2800 mosM, about 2900 mosM, or about 3000 mosM. In certain embodiments 0.5 ml-20 ml of the hyperosmotic solution may be administered such as about 0.5 ml about 1 ml, about 2 ml, about 3 ml, about 4 ml about 5 ml about 6 ml about 7 ml about 8 ml about 9 ml about 10 ml about 11 ml, about 12 ml about 13 ml about 14 ml about 15 ml about 16 ml about 17 ml about 18 ml about 19 m1 or about 20 m1. In one embodiment 4 ml of 2400 mosM solution of the hyperosmotic agent is administered as a nebulized aerosol using a nebuliser such as a jet or ultrasonic nebulizer.

In another embodiment the increase in the osmolarity in a recipient's lung is achieved by administration of a hyperosmotic agent as a dry powder inhaled formulation. In one embodiment 0.5 mosmoles-3.0 mosmoles, 1 mosmoles-2.5 mosmoles or 1.5 mosmoles-2 mosmoles, or about 0.5 mosmoles, about 0.6 mosmoles, about 0.7 mosmoles, about 0.8 mosmoles, about 0.9 mosmoles, about 1.0 mosmoles, about 1.1 mosmoles, about 1.2 mosmoles, about 1.3 mosmoles, about 1.4 mosmoles, about 1.5 mosmoles, about 1.6 mosmoles, about 1.7 mosmoles, about 1.8 mosmoles, about 1.9 mosmoles, about 2.0 mosmoles, about 2.1 mosmoles, about 2.2 mosmoles, about 2.3 mosmoles, about 2.4 mosmoles, about 2.5 mosmoles, about 2.6 mosmoles, about 2.7 mosmoles, about 2.8 mosmoles, about 2.9 mosmoles, or about 3.0 mosmoles of a hyperosmotic agent is administered as a dry powder inhaled formulation. In one embodiment 2.2 mosmoles of a hyperosmotic agent is administered as a dry powder inhaled formulation.

In one embodiment the hyperosmotic agent is hypertonic saline. The hypertonic saline may have an osmolarity of 600 mosM-3000 mosM, such as 700 mosM-2900 mosM, 800 mosM-2800 mosM, 900 mosM-2700 mosM, 1000 mosM-2600 mosM, 1000 mosM-2500 mosM, 1100 mosM-2500 mosM, 1200 mosM-2500 mosM, 1300 mosM-2500 mosM, 1400 mosM-2500 mosM, 1500 mosM-2500 mosM, 1600 mosM-2500 mosM, 1700 mosM-2500 mosM, 1800 mosM-2500 mosM, 1900 mosM-2500 mosM, 2000 mosM-2500 mosM, or about 600 mosM, about 700 mosM, about 800 mosM, about 900 mosM, about 1000 mosM, about 1100 mosM, about 1200 mosM, about 1300 mosM, about 1400 mosM, about 1500 mosM, about 1600 mosM, about 1700 mosM, about 1800 mosM, about 1900 mosM, about 2000 mosM, about 2100 mosM, about 2200 mosM, about 2300 mosM, about 2400 mosM, about 2500 mosM, about 2600 mosM, about 2700 mosM, about 2800 mosM, about 2900 mosM, or about 3000 mosM. In certain embodiments 0.5 ml-20 ml of the hyperosmotic saline solution may be administered such as about 0.5ml about 1 ml, about 2 ml, about 3 ml, about 4 ml about 5 ml about 6 ml about 7 ml about 8 ml about 9 ml about 10 ml about 11 ml, about 12 ml about 13 ml about 14 ml about 15 ml about 16 ml about 17 ml about 18 ml about 19 ml or about 20 ml. In one embodiment 4 ml of 2400 mosM hyperosmotic saline solution is administered as a nebulized aerosol using a jet or ultrasonic nebulizer.

In another embodiment the hyperosmotic agent is mannitol. Within this embodiment, the hyperosmotic mannitol solution may be administered as a nebulized aerosol using a jet or ultrasonic nebulizer. The hyperosmotic mannitol solution may have an osmolarity of 600 mosM -3000 mosM, such as 700 mosM-2900 mosM, 800 mosM-2800 mosM, 900 mosM-2700 mosM, 1000 mosM-2600 mosM, 1000 mosM-2500 mosM, 1100 mosM-2500 mosM, 1200 mosM-2500 mosM, 1300 mosM-2500 mosM, 1400 mosM-2500 mosM, 1500 mosM-2500 mosM, 1600 mosM-2500 mosM, 1700 mosM-2500 mosM, 1800 mosM-2500 mosM, 1900 mosM-2500 mosM, 2000 mosM-2500 mosM, or about 600 mosM, about 700 mosM, about 800 mosM, about 900 mosM, about 1000 mosM, about 1100 mosM, about 1200 mosM, about 1300 mosM, about 1400 mosM, about 1500 mosM, about 1600 mosM, about 1700 mosM, about 1800 mosM, about 1900 mosM, about 2000 mosM, about 2100 mosM, about 2200 mosM, about 2300 mosM, about 2400 mosM, about 2500 mosM, about 2600 mosM, about 2700 mosM, about 2800 mosM, about 2900 mosM, or about 3000 mosM. In certain embodiments 0.5 ml-12 ml of the hyperosmotic mannitol solution may be administered such as about 0.5 ml about 1 ml, about 2 ml, about 3 ml, about 4 ml about 5 ml about 6 ml about 7 ml about 8 ml about 9 ml about 10 ml about 11 ml, or about 12 ml. In one embodiment 4 ml of 2400 mosM hyperosmotic mannitol solution is administered as a nebulized aerosol using a jet or ultrasonic nebulizer.

In a further embodiment mannitol may be administered as a dry powder inhaled formulation. The mannitol dry powder inhaled formulation may have an osmolarity of 0.5 mosmoles-3.0 mosmoles, such as 1.0 mosmoles-2.5 mosmoles, 1.5 mosmoles-2.5 mosmoles, 2.0 mosmoles-2.5 mosmoles, or about 0.5 mosmoles, about 0.6 mosmoles, about 0.7 mosmoles, about 0.8 mosmoles, about 0.9 mosmoles, about 1.0 mosmoles, about 1.1 mosmoles, about 1.2 mosmoles, about 1.3 mosmoles, about 1.4 mosmoles, about 1.5 mosmoles, about 1.6 mosmoles, about 1.7 mosmoles, about 1.8 mosmoles, about 1.9 mosmoles, about 2.0 mosmoles, about 2.1 mosmoles, about 2.2 mosmoles, about 2.3 mosmoles, about 2.4 mosmoles, about 2.5 mosmoles, about 2.6 mosmoles, about 2.7 mosmoles, about 2.8 mosmoles, about 2.9 mosmoles, or about 3.0 mosmoles.

In a further embodiment the hyperosmotic agent is sodium gluconate. Within this embodiment, the sodium gluconate may be administered as a nebulized aerosol using a jet or ultrasonic nebulizer. The sodium gluconate solution may have an osmolarity of 600 mosM-3000 mosM, such as 700 mosM-2900 mosM, 800 mosM-2800 mosM, 900 mosM-2700 mosM, 1000 mosM-2600 mosM, 1000 mosM-2500 mosM, 1100 mosM-2500 mosM, 1200 mosM-2500 mosM, 1300 mosM-2500 mosM, 1400 mosM-2500 mosM, 1500 mosM-2500 mosM, 1600 mosM-2500 mosM, 1700 mosM-2500 mosM, 1800 mosM-2500 mosM, 1900 mosM-2500 mosM, 2000 mosM-2500 mosM, or about 600 mosM, about 700 mosM, about 800 mosM, about 900 mosM, about 1000 mosM, about 1100 mosM, about 1200 mosM, about 1300 mosM, about 1400 mosM, about 1500 mosM, about 1600 mosM, about 1700 mosM, about 1800 mosM, about 1900 mosM, about 2000 mosM, about 2100 mosM, about 2200 mosM, about 2300 mosM, about 2400 mosM, about 2500 mosM, about 2600 mosM, about 2700 mosM, about 2800 mosM, about 2900 mosM, or about 3000 mosM. In certain embodiments 0.5 ml-20 ml of the hyperosmotic sodium gluconate solution may be administered such as about 0.5 ml about 1 ml, about 2 ml, about 3 ml, about 4 ml about 5 ml about 6 ml about 7 ml about 8 ml about 9 ml about 10 ml about 11 ml, about 12 ml about 13 ml about 14 ml about 15 ml about 16 ml about 17 ml about 18 ml about 19 ml or about 20 ml. In one embodiment sodium gluconate may be administered as 4 ml of 2400 mosM hyperosmotic sodium gluconate solution as a nebulized aerosol using a jet or ultrasonic nebulizer.

In a further embodiment sodium gluconate may be administered as a dry powder inhaled formulation. The sodium gluconate dry powder inhaled formulation may have an osmolarity of 0.5 mosmoles-3.0 mosmoles, such as 1.0 mosmoles-2.5 mosmoles, 1.5 mosmoles-2.5 mosmoles, 2.0 mosmoles-2.5 mosmoles, or about 0.5 mosmoles, about 0.6 mosmoles, about 0.7 mosmoles, about 0.8 mosmoles, about 0.9 mosmoles, about 1.0 mosmoles, about 1.1 mosmoles, about 1.2 mosmoles, about 1.3 mosmoles, about 1.4 mosmoles, about 1.5 mosmoles, about 1.6 mosmoles, about 1.7 mosmoles, about 1.8 mosmoles, about 1.9 mosmoles, about 2.0 mosmoles, about 2.1 mosmoles, about 2.2 mosmoles, about 2.3 mosmoles, about 2.4 mosmoles, about 2.5 mosmoles, about 2.6 mosmoles, about 2.7 mosmoles, about 2.8 mosmoles, about 2.9 mosmoles, or about 3.0 mosmoles.

The Purinergic Agonist

In one embodiment the amidino compound or Amiloride-sensitive sodium channel (ENaC) regulator described above is used with a purinergic agonist.

In another embodiment, the purinergic agonist may be uridine-5′-triphosphate (UTP), P¹, P⁴-bis(5′-uridyl) tetraphosphate tetrasodium salt (Diquafosol) or 2′-deoxycytidine(5′) tetraphospho (5′) uridine tetrasodium salt (Denufosol). The purinergic agonist may be administered at a dose of up to 60 mg, such as up to 50 mg, up to 40 mg, up to 30 mg, up to 20 mg or up to 10 mg, including about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60mg. In one embodiment the purinergic agonist may be administered by inhalation.

Role of ENaC Regulating Substances in Increasing Ciliary Transport of Mucus Secretions

The present inventors have demonstrated that substances that block or down regulate airway ENaC activity also act to increase ciliary transport of mucus secretions in a model in the trachea. Without wishing to be bound by theory, the inventors consider that it is the differential effect of 6-amidino-2-napthyl 4-guanidinobenzoate and hyperosmotic agents or purinergic agonists on ciliary transport of mucus secretions which permits the enhancement of the ENaC inhibitory effect demonstrated when the active agents are used in combination.

The inventors have demonstrated that ENaC regulating substances fit into three distinct classes of ciliary transport enhancers.

Class 1: Rapid onset—short duration. Amiloride and other exofacial sodium channel blockers are members of this class. Onset of increased ciliary transport of secretions is immediate. The peak increase in the rate of ciliary transport is 30%, and this is achieved at 15 to 30 min following application of the substance to the trachea. The duration of elevated ciliary transport rate is short, returning to control transport rate by 40 min.

Class 2: Medium onset—medium duration. Hypertonic saline, hyperosmotic mannitol, hyperosmotic sodium gluconate, and other hyperosmotic treatments fit to this class. UTP and other purinergic agonists are also members of this class. Increased ciliary transport of secretions is observed at the first measured time point 25 min after application of the treatment to the trachea. The peak increase in the rate of ciliary transport is 40 to 50%, and this is achieved at 1.5 to 2 h. The duration of elevated ciliary transport is medium, returning to control transport rate by 4 h.

Class 3: Slow onset—long duration. 6-amidino-2-napthyl 4-guanidinobenzoate and related compounds fit to this class. Kunitz-type serine protease inhibitors also fit to this class. The first time point where increased ciliary transport of secretions is observed is at 55 min after application of the treatment. The peak increase in the rate of ciliary transport is 70 to 100%, and this is achieved at 3.5 to 5.5 h. Elevated ciliary transport is long duration, it continues to exceed control transport rate at 24 h following exposure to the treatment.

By way of explanation, but not limitation, the inventors consider that the use of a slow onset—long duration ENaC regulating substance (e.g. 6-amidino-2-napthyl 4-guanidinobenzoate and related compounds) in combination with a medium onset-medium duration ENaC regulating substance (e.g. a hyperosmotic agent or a purinergic agonist) leads to the observed enhanced ENaC activity inhibitory effect.

Respiratory Diseases

The intended respiratory diseases include diseases of the lung, airways (bronchioles, bronchi), and upper respiratory tract (nose, para-nasal sinuses, Eustachian tube and middle ear) but are not limited to those diseases that are characterised by poor mucociliary clearance and/or mucostasis.

Preferably the disease is selected from the group comprising cystic fibrosis, bronchiectasis, chronic bronchitis, chronic obstructive pulmonary disease (COPD), rhino-sinusitis and otitis media.

Preferably the disease is selected from the group consisting of cystic fibrosis, bronchiectasis, chronic bronchitis, chronic obstructive pulmonary disease (COPD), rhino-sinusitis and otitis media.

Preferably, the respiratory disease is cystic fibrosis.

Additional Therapeutic Agents, Carrier and Diluents

In one embodiment, the amidino compound or other Amiloride-sensitive sodium channel (ENaC) regulator and/or the hyperosmotic agent or purinergic agonist further comprises one or more pharmaceutically acceptable carriers or diluents. Herein, and throughout, the term “and/or” indicates that either one or both of the active ingredients may include the additional feature. In this case specifically, either one or both of the active ingredients may further comprise one or more pharmaceutically acceptable carriers or diluents. Where the amidino compound or other Amiloride-sensitive sodium channel (ENaC) regulator and the hyperosmotic agent or purinergic agonist are present in a single composition this term indicates that the composition includes the additional feature. In this case specifically a composition comprising the amidino compound or other Amiloride-sensitive sodium channel (ENaC) regulator and the hyperosmotic agent or purinergic agonist may comprise one or more pharmaceutically acceptable carriers or diluents.

In another embodiment, the amidino compound or other Amiloride-sensitive sodium channel (ENaC) regulator and/or the hyperosmotic agent or purinergic agonist may be in aqueous solution.

In a further embodiment the amidino compound or other Amiloride-sensitive sodium channel (ENaC) regulator and/or the hyperosmotic agent or purinergic agonist also comprises one or more other active agents selected from the group of antibiotics, vaccines, decongestants (nasal or bronchial), rhDNase, non-steroidal antiinflamatory agents (NSAIDs), steroids, antiviral agents, elastase inhibitors, exofacial sodium channel blocking agents, gene therapy agents, chloride channel activators and bronchodilators.

Combined, Separate or Sequential Administration

Throughout all uses and methods of the invention, the amidino compound or other Amiloride-sensitive sodium channel (ENaC) regulator and the hyperosmotic agent or purinergic agonist may be for combined, separate or sequential administration.

Combined administration indicates that the amidino compound or other Amiloride-sensitive sodium channel (ENaC) regulator and the hyperosmotic agent or purinergic agonist are administered together as a single composition.

Separate administration indicates that the amidino compound or other Amiloride-sensitive sodium channel (ENaC) regulator and the hyperosmotic agent or purinergic agonist are administered individually. In this embodiment the amidino compound or other Amiloride-sensitive sodium channel (ENaC) regulator and the hyperosmotic agent or purinergic agonist may be administered at the same time or at different times, but are administered as individual compositions.

Sequential administration indicated that the amidino compound or other Amiloride-sensitive sodium channel (ENaC) regulator and the hyperosmotic agent or purinergic agonist are administered separately, in either order, with a temporal separation between administration of the two active agents. Herein the amidino compound or other Amiloride-sensitive sodium channel (ENaC) regulator may be administered before or after the hyperosmotic agent or purinergic agonist and the hyperosmotic agent or purinergic agonist may be administered before or after the amidino compound or other Amiloride-sensitive sodium channel (ENaC) regulator. In certain embodiments administration of the two active agents may be separated by 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 40 minutes, 50 minutes, 60 minutes, 90 minutes, 2 hours, or 3 hours.

Methods of Administration

In one embodiment, the amidino compound or other Amiloride-sensitive sodium channel (ENaC) regulator and/or the hyperosmotic agent or purinergic agonist may be formulated for administration to the respiratory system by the pulmonary route.

Preferably, the amidino compound or other Amiloride-sensitive sodium channel (ENaC) regulator and/or the hyperosmotic agent or purinergic agonist is formulated for administration by a method including but not limited to intratracheal installation (delivery of solution into the airways by syringe), intratracheal delivery of liposomes, insufflation (administration of powder formulation by syringe or any other similar device into the airways), nebulization, dry powder inhalation and aerosol inhalation.

Aerosols (e.g. jet or ultrasonic nebulizers, metered-dose inhalers (MDIs), and dry-powder inhalers (DPIs) can also be used in intranasal applications as well as for pulmonary administration.

Aerosol formulations are stable dispersions or suspensions of solid material and liquid droplets in a gaseous medium and can be placed into pressurized acceptable propellants, such as hydrofluoroalkanes (HFAs, i.e. HFA-134a and HFA-227, or a mixture thereof), dichlorodifluoromethane (or other chlorofluorocarbon propellants such as a mixture of Propellants 11,12, and/or 114), propane, nitrogen, and the like.

Preferably the amidino compound or other Amiloride-sensitive sodium channel (ENaC) regulator and/or the hyperosmotic agent or purinergic agonist is formulated for administration by dry powder inhalation or aerosol inhalation together with a propellant selected from the group comprising hydrofluoroalkanes, chlorofluorocarbons, propane, nitrogen, or a mixture thereof. Alternatively, the propellant may be selected from the group consisting of hydrofluoroalkanes, chlorofluorocarbons, propane, nitrogen, or a mixture thereof.

In preferred embodiments the amidino compound or other Amiloride-sensitive sodium channel (ENaC) regulator and/or the hyperosmotic agent or purinergic agonist of the invention are formulated for the treatment of the nasal epithelium, para-nasal sinuses, the Eustachian tube or middle ear, and are formulated as solutions for delivery as drops, by pipette or by syringe or by aerosol to the nasal epithelium, para-nasal sinuses, the Eustachian tube or middle ear.

In further embodiments for the treatment of rhino-sinusitis, the amidino compound or other Amiloride-sensitive sodium channel (ENaC) regulator and/or the hyperosmotic agent or purinergic agonist is formulated as a solution for the irrigation of the para-nasal sinuses by local installation of said solution via cannula tube or syringe needle or by nasal aerosol.

In another embodiment for the treatment of otitis media, the amidino compound or other

Amiloride-sensitive sodium channel (ENaC) regulator and/or the hyperosmotic agent or purinergic agonist is formulated as a solution for delivery as solution directly applied to the middle ear via the external auditory meatus and canal or by nasal aerosol.

Composition

In another aspect the invention is directed to a composition comprising an amidino compound of the general formula (I):

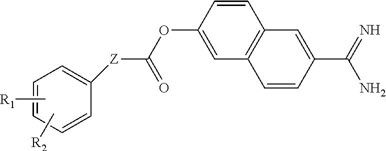

and a hyperosmotic agent or a purinergic agonist.

The composition is preferably formulated for delivery to the respiratory system and may be administered in accordance with any of the methods of administration discussed above.

The composition may be for use in a method of treating respiratory disease.

It will be apparent to the skilled person that a composition comprising both the amidino compound and a hyperosmotic agent or a purinergic agonist will necessarily require these two active ingredients to be administered together.

Inhalation Device

The current invention further relates to an inhalation device loaded with a pharmaceutical composition of the current invention. Preferably, the inhalation device is a dry powder inhaler, metered dose inhaler, jet nebulizer, ultrasonic nebulizer, or nebulizer for nasal delivery.

### EXAMPLES

The invention will be now be described by way of exemplification only with reference to the following Examples.

The present inventors have found 6-amidino-2-napthyl 4-guanidinobenzoate in combination with a hyperosmotic agent or a purinergic agonist to be a potent long duration inhibitor of ENaC activity in airway epithelial cells. The inventors consider that the enhanced activity of 6-amidino-2-napthyl 4-guanidinobenzoate in combination with a hyperosmotic agent or a purinergic agonist is likely due to the demonstrated ability of ENaC regulatory agents to increase ciliary transport of respiratory mucus secretions. In combination these two active ingredients demonstrate activities which exceed the activity of either active ingredient alone. This and related compounds therefore have potential in the treatment of respiratory diseases characterised by mucostasis such as cystic fibrosis, bronchiectasis, chronic bronchitis, chronic obstructive pulmonary disease (COPD), rhino-sinusitis and otitis media. The pharmacological profile of 6-amidino-2-napthyl 4-guanidinobenzoate in combination with a hyperosmotic agent or a purinergic agonist is not predicted by previous studies.

The preferred respiratory disease for treatment with such compounds is cystic fibrosis.

The term “respiratory diseases” includes but is not limited to conditions of the lung, airways (bronchioles, bronchi), and upper respiratory tract (nose, para-nasal sinuses, Eustachian tube and middle ear) characterised by impaired mucociliary clearance, possibly resulting in mucostasis.

Such conditions include but are not limited to cystic fibrosis, bronchiectasis, chronic bronchitis, chronic obstructive pulmonary disease (COPD), rhino-sinusitis and otitis media.

The current invention will be further understood by way of the following examples.

General Methods

The following examples use the same set of material and methods, which are described below.

Measurement of Tracheal PD

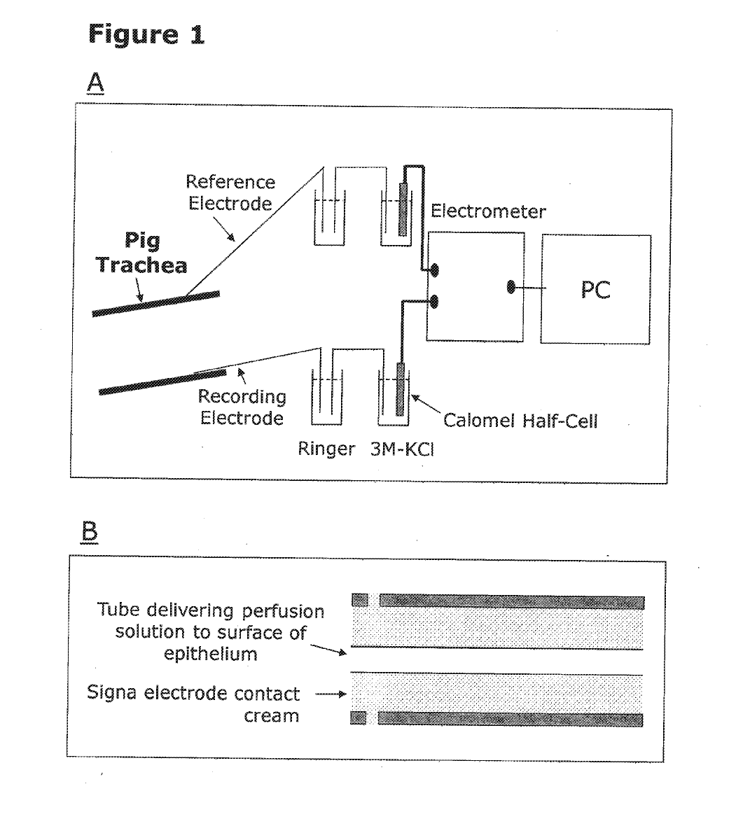

Pig tracheas were obtained from a local abattoir or Veterinary University. The lumen of each trachea was cleaned by gentle irrigation with BSS. A reference electrode consisting of silicone tubing filled with Signa electrode cream was attached to the outer surface of the trachea according to the diagram in FIG. 1A. Pig tracheas were placed on a sloping radiator heated at 37° C. A double lumen recording electrode (FIG. 1B) was placed inside the trachea in contact with the tracheal epithelium. The inner tubing of the recording electrode was used to perfuse onto the surface of the trachea buffer solutions and buffer solutions containing drugs and agents. The outer part of the recording electrode was also filled with Signa electrode cream. The ends of the electrodes not in contact with the trachea were placed in Ringer buffer solution. Electrical contact to calomel half-cells in 3M-KCl solution was achieved by silicone tubing bridges filled with Signa electrode cream. The calomel half-cells were connected to a high impedance digital electrometer (Agilent U1252A). Digital PD values were recorded continuously every 1 second for the duration of each study and were filed onto a PC.

During each study the amiloride-sensitive, ENaC-dependent component of tracheal PD was determined periodically by perfusion (at 1 ml/min) of buffer solution containing 3×10⁻⁶ M amiloride onto the epithelial surface of the trachea for 5 min, followed by washout for 10 minutes with buffer solution without amiloride. Each response to amiloride was quantified by measuring the area under the curve (AUC) response (mV.min) to amiloride perfusion and washout.

Ciliary Transport of Mucus Secretions

Pig tracheas (from larynx to carina) from normal wild type animals were obtained from a local abattoir or Veterinary University. Transgenic CF piglets were produced as described by Klymiuk et al. 2012, and tracheas were obtained at a Veterinary University. The lumen of each trachea was cleaned by gentle irrigation with BSS. Tracheas were opened longitudinally with scissors along the posterior aspect and mounted with the posterior aspect upwards in a moist air-tight box. An aliquot of pig airway mucus approximately 3 mm diameter was placed on the mucosal surface close to the distal end of each trachea. Tracheal mucus velocity (TMV mm/min) was measured by recording the distance moved, towards the laryngeal end of the trachea, in a given time. At the start of a study three measurements of TMV were made for each trachea to establish a baseline rate for the trachea. Solutions were then pipetted onto the luminal mucosal surface of tracheas for 10 min. Then excess solution was tipped off the mucosal surface and measurement of TMV repeated. Further measurements of TMV were at later times as indicated in FIGS. 10 to 14. TMV results at each time point are expressed as % of the baseline TMV, ciliary transport index (mean±SD).

### Example 1

### Down-Regulation of Amiloride-Sensitive ENaC-Dependent Tracheal PD by 6-Amidino-2-Napthyl 4-Guanidinobenzoate

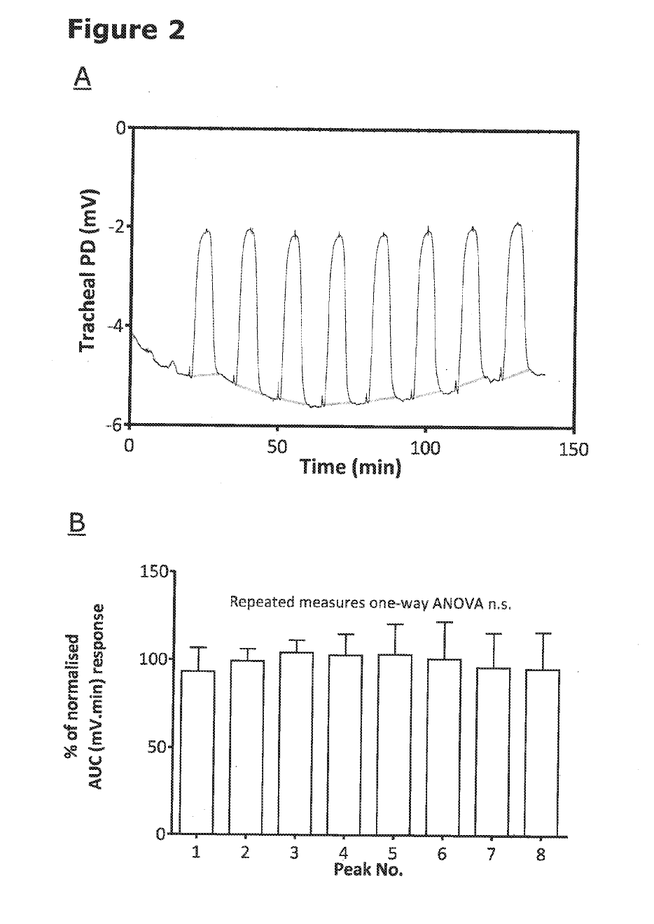

FIG. 2A shows the amiloride-sensitive tracheal PD response in ex vivo pig trachea perfused at 1 ml/min with balanced salt solution (BSS) containing 0.145M-NaCl, 5 mM-NaHCO₃, 4.05mM-KCl, 1.2 mM-CaCl₂, 1.2 mM-MgCl₂, 2.4 mM-K₂HPO₄, 0.4 mM-KH₂PO₄, 10 mM glucose adjusted to pH7.4. PD values were recorded every 1 s for 140 min. At 20, 35, 50, 65, 80, 95, 110 and 125 min 3×10⁶M-amiloride was added to the perfusing BSS for 5 min; followed by 10 min wash out with BSS alone. FIG. 2A is the trace of the mean data from recordings from 23 pig tracheas. In response to perfusion of amiloride tracheal PD depolarises rapidly to reach a plateau level about 2 min following initiation of amiloride perfusion. After each period of perfusion of amiloride tracheal PD repolarised and returned to baseline approximately 5 min after initiation of washout.

FIG. 2B shows the area under the curve (AUC) response (mean+SD) to amiloride perfusion for each of the eight peaks in FIG. 2A. Repeated measures one-way ANOVA showed no significant differences in the amiloride stimulation and washout responses repeated over 140 min. AUC data is normalised by expressing the response for each peak as the percentage of the average response for the first four peaks. The data demonstrates that the method can be used to study the effects of other drugs and agents on the amiloride-sensitive ENaC-dependent tracheal PD response.

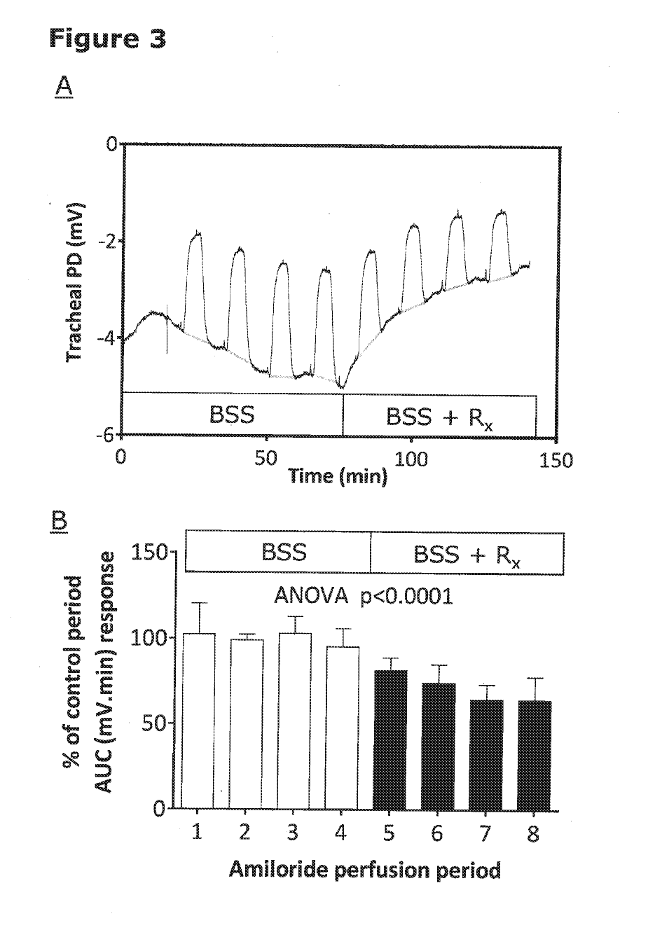

FIGS. 3A and 3B illustrate the effect of perfusion of the ENaC down-regulating drug 6-amidino-2-napthyl 4-guanidinobenzoate (Rₓ) on amiloride-sensitive tracheal PD responses in the ex vivo pig trachea. Tracheas were perfused with BSS between 0 to 75 min, followed by perfusion with BSS containing 3.10⁻⁵M 6-amidino-2-napthyl 4-guanidinobenzoate. PD values were recorded every 1 s for 140 min. At 20, 35, 50, 65, 80, 95, 110 and 125 min 3×10⁻⁶M-amiloride was added to the perfusing BSS or BSS+Rₓ for 5 min; followed by 10 min wash out with buffer. FIG. 3A is the trace of the mean data from recordings from 8 pig tracheas. Perfusion with 6-amidino-2-napthyl 4-guanidinobenzoate caused the baseline tracheal PD to depolarise, and the responses to 5 min amiloride perfusion to decrease. FIG. 3B shows the area under the curve (AUC) response (mean+SD) to amiloride perfusion for each of the eight peaks in FIG. 3A. AUC data is normalised by expressing the response for each peak as the percentage of the average response for the first four peaks. Analysis of AUC responses by one-way ANOVA confirmed significant reduction (p<0.0001) in the AUC responses in response to perfusion of 6-amidino-2-napthyl 4-guanidinobenzoate.

Down-regulation of amiloride-sensitive ENaC-dependent tracheal PD responses were further studied at doses of 6-amidino-2-napthyl 4-guanidinobenzoate of 3.10⁸ M, 10⁷ M, 3.10⁷ M, 10⁶ M, 3.10⁻⁶ M, and 10⁻⁵ M. Significant down-regulation of amiloride-sensitive ENaC-dependent tracheal PD responses was observed at all doses.

### Example 2

### Hyperosmotic Saline Down-Regulates Amiloride-Sensitive ENaC-Dependent Tracheal PD; and Enhances Down-Regulation by 6-Amidino-2-Napthyl 4-Guanidinobenzoate

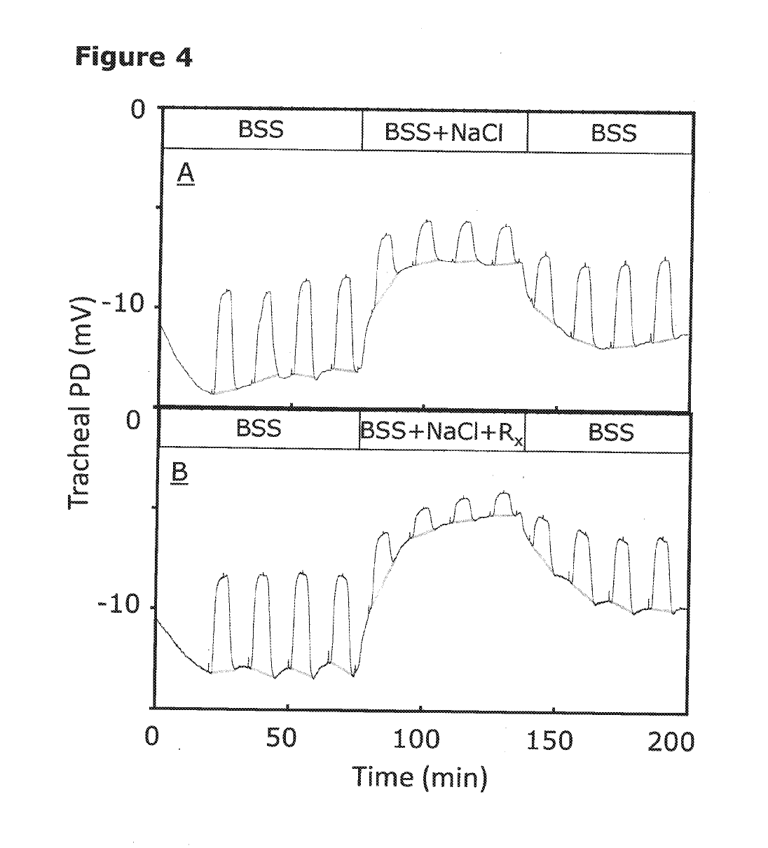

FIG. 4A illustrates the effect of perfusion of hyperosmotic saline (NaCl) on amiloride-sensitive ENaC-dependent tracheal PD responses in ex vivo pig tracheas. Tracheas were perfused with BSS between 0 to 75 min, followed by perfusion between 75 min and 140 min with BSS containing a further 50 mM-NaCl (100 mosM-NaCl), then finally perfused between 140 min and 200 min with BSS. PD values were recorded every 1 s for 200 min. At 20, 35, 50, 65, 80, 95, 110, 125, 140, 155, 170 and 185 min 3×10⁻⁶M-amiloride was added to the perfusing BSS or BSS+NaCl for 5 min; followed by 10 min wash out with buffer. FIG. 4A is the trace of the mean data from recordings from 15 pig tracheas. Perfusion with BSS plus 50 mM-NaCl caused the baseline tracheal PD to depolarise, and the responses to 5 min amiloride perfusion to decrease. Perfusion from 140 to 200 min with BSS without added NaCl caused the baseline tracheal PD to partially repolarise, and the responses to 5 min amiloride perfusion to recover.

FIG. 4B shows the effect of perfusion of a combination of hyperosmotic saline (NaCl) and 6-amidino-2-napthyl 4-guanidinobenzoate (Rₓ) on amiloride-sensitive ENaC-dependent tracheal PD responses in ex vivo pig tracheas. Tracheas were perfused with BSS between 0 to 75 min, followed by perfusion between 75 min and 140 min with BSS containing a further 50 mM-NaCl (100 mosM-NaCl) and 10⁻⁵ M 6-amidino-2-napthyl 4-guanidinobenzoate, then finally perfused between 140 min and 200 min with BSS. PD values were recorded every 1 s for 200 min. At 20, 35, 50, 65, 80, 95, 110, 125, 140, 155, 170 and 185 min 3×10⁻⁶M-amiloride was added to the perfusing BSS or BSS+NaCl+Rₓ for 5 min; followed by 10 min wash out with buffer. FIG. 4B is the trace of the mean data from recordings from 17 pig tracheas. Perfusion with BSS plus 50 mM-NaCl plus 6-amidino-2-napthyl 4-guanidinobenzoate caused the baseline tracheal PD to depolarise, and the responses to 5 min amiloride perfusion to decrease. Perfusion from 140 to 200 min with BSS without added NaCl caused the baseline tracheal PD to partially repolarise, and the responses to 5 min amiloride perfusion to recover.

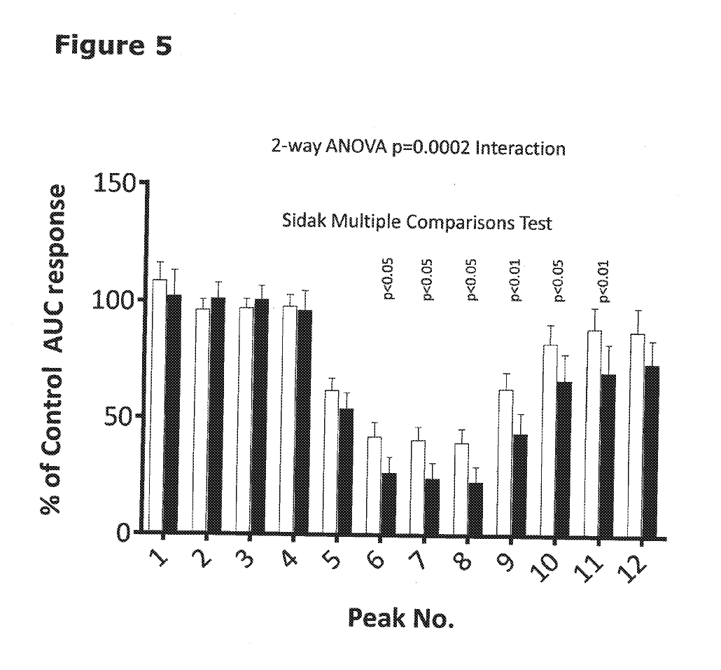

AUC analysis of the effects of perfusion of hyperosmotic saline (NaCl) (open columns), and the combination of hyperosmotic saline plus 6-amidino-2-napthyl 4-guanidinobenzoate (filled columns) is shown in FIG. 5. Two-way analysis of variance of AUC confirmed that both hyperosmotic saline and the combination significantly reduced the amiloride-sensitive ENaC-dependent tracheal PD responses, and that reduced responses were significantly greater for treatment with the combination than with hyperosmotic saline alone.

### Example 3

### Hyperosmotic Mannitol Down-Regulates Amiloride-Sensitive ENaC-Dependent Tracheal PD; and Enhances Down-Regulation by 6-Amidino-2-Napthyl 4-Guanidinobenzoate

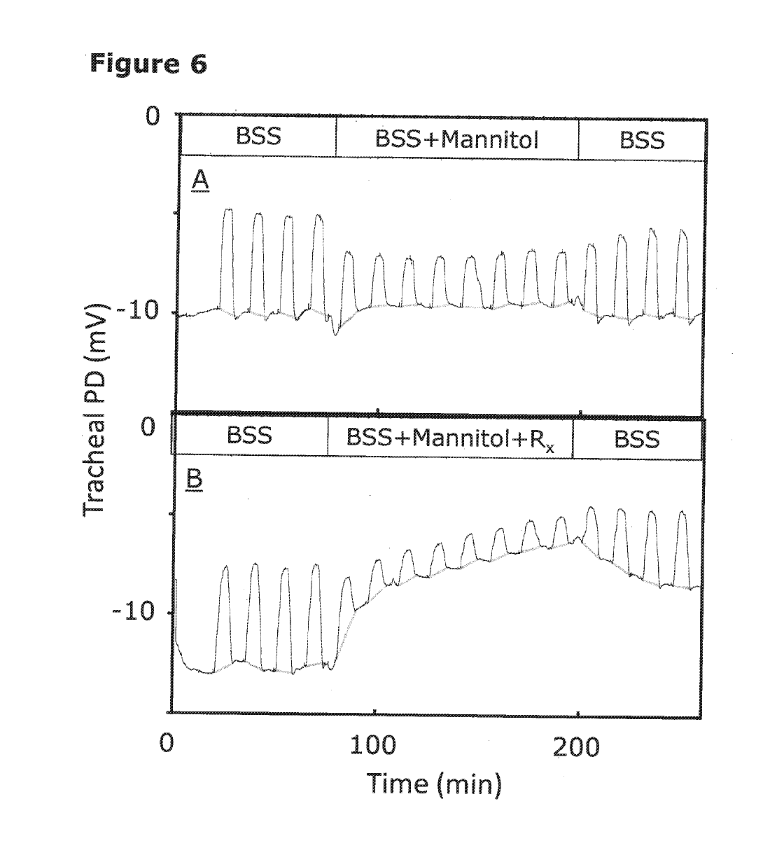

FIG. 6A illustrates the effect of perfusion of hyperosmotic mannitol on amiloride-sensitive ENaC-dependent tracheal PD responses in ex vivo pig tracheas. Tracheas were perfused with BSS between 0 to 75 min, followed by perfusion between 75 min and 200 min with BSS containing a 100 mosM-mannitol, then finally perfused between 200 min and 260 min with BSS. PD values were recorded every 1 s for 200 min. At 20, 35, 50, 65, 80, 95, 110, 125, 140, 155, 170, 185, 200, 215, 230 and 245 min 3×10⁻⁶M-amiloride was added to the perfusing BSS or BSS+mannitol for 5 min; followed by 10 min wash out with buffer. FIG. 6A is the trace of the mean data from recordings from 17 pig tracheas. Perfusion with BSS plus 100 mosM-mannitol caused the responses to 5 min amiloride perfusion to decrease. Perfusion from 200 to 260 min with BSS without added mannitol caused the responses to 5 min amiloride perfusion to recover.

FIG. 6B shows the effect of perfusion of a combination of hyperosmotic mannitol and 6-amidino-2-napthyl 4-guanidinobenzoate (Rₓ) on amiloride-sensitive ENaC-dependent tracheal PD responses in ex vivo pig tracheas. Tracheas were perfused with BSS between 0 to 75 min, followed by perfusion between 75 min and 200 min with BSS containing 100 mosM-mannitol and 10⁻⁵ M 6-amidino-2-napthyl 4-guanidinobenzoate, then finally perfused between 200 min and 260 min with BSS. PD values were recorded every 1 s for 200 min. At 20, 35, 50, 65, 80, 95, 110, 125, 140, 155, 170, 185, 200, 215, 230 and 245 min 3×10⁻⁶M-amiloride was added to the perfusing BSS or BSS+mannitol+Rₓ for 5 min; followed by 10 min wash out with buffer. FIG. 6B is the trace of the mean data from recordings from 18 pig tracheas. Perfusion with BSS plus 100 mosM-mannitol plus 6-amidino-2-napthyl 4-guanidinobenzoate caused the baseline tracheal PD to depolarise, and the responses to 5 min amiloride perfusion to decrease. Perfusion from 200 to 260 min with BSS without added mannitol caused the baseline tracheal PD to partially repolarise, and the responses to 5 min amiloride perfusion to recover.

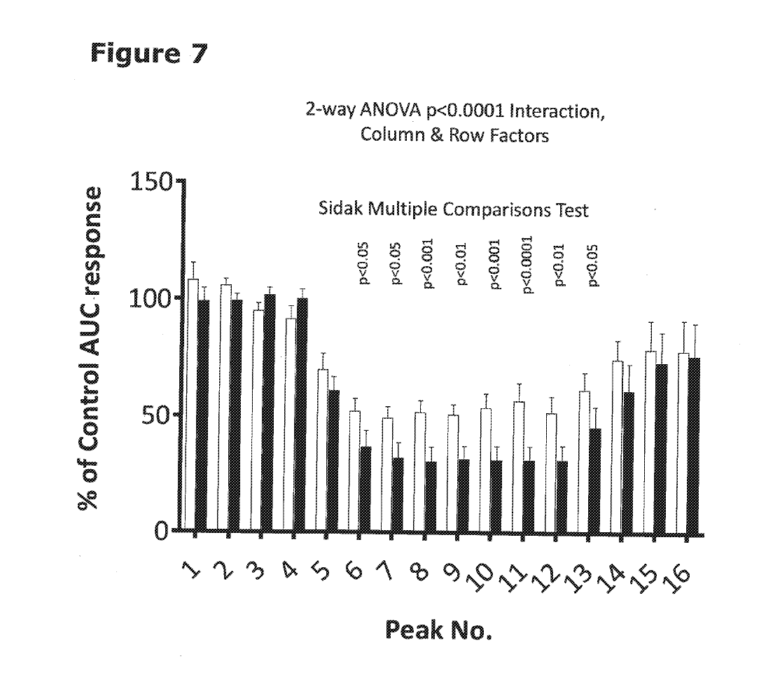

AUC analysis of the effects of perfusion of hyperosmotic mannitol (open columns), and the combination of hyperosmotic mannitol plus 6-amidino-2-napthyl 4-guanidinobenzoate (filled columns) is shown in FIG. 7. Two-way analysis of variance of AUC confirmed that both hyperosmotic mannitol and the combination significantly reduced the amiloride-sensitive ENaC-dependent tracheal PD responses, and that reduced responses were significantly greater for treatment with the combination than with hyperosmotic mannitol alone.

### Example 4

### Hyperosmotic Sodium Gluconate Down-Regulates Amiloride-Sensitive ENaC-Dependent Tracheal PD

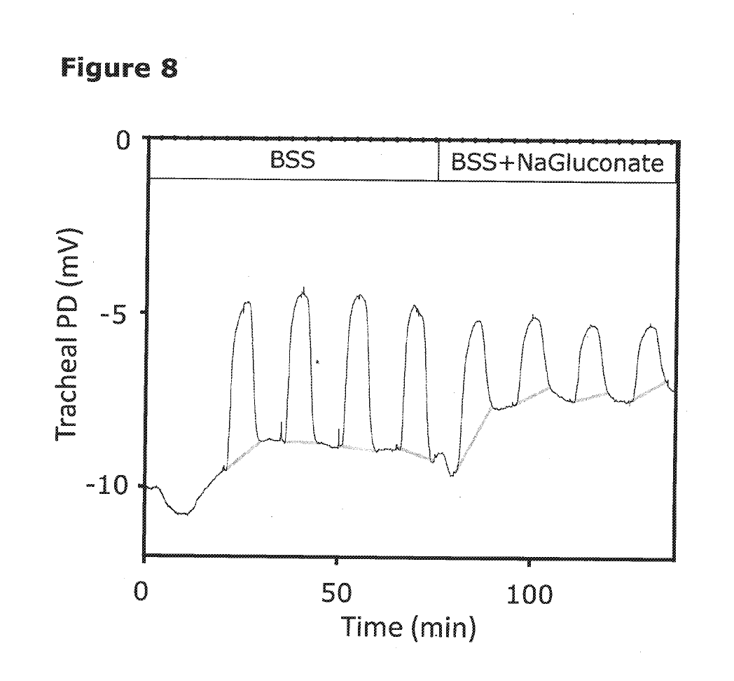

FIG. 8 illustrates the effect of hyperosmotic sodium gluconate on amiloride-sensitive ENaC-dependent tracheal PD. Pig tracheas were perfused with BSS between 0 to 75 min, followed by perfusion with BSS containing 50 mM (100 mosM) sodium gluconate. PD values were recorded every 1 s for 140 min. At 20, 35, 50, 65, 80, 95, 110 and 125 min 3×10⁻⁶ M-amiloride was added to the perfusing BSS or BSS+NaGluconate for 5 min; followed by 10 min wash out with buffer. FIG. 8 is the trace of the mean data from recordings from 6 pig tracheas. Perfusion with hyperosmotic sodium gluconate caused the baseline tracheal PD to partially depolarise, and the responses to 5 min amiloride perfusion to decrease.

### Example 5

### UTP Down-Regulates Amiloride-Sensitive ENaC-Dependent Tracheal PD

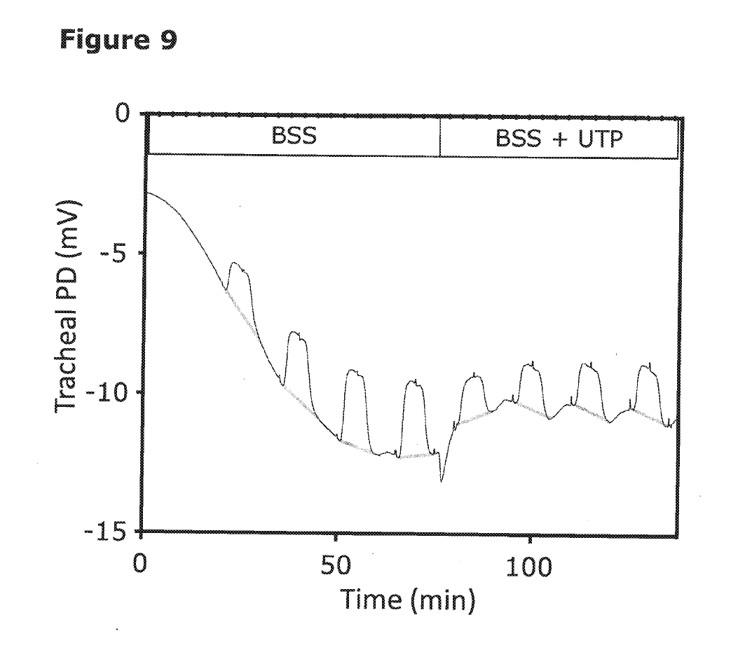

FIG. 9 shows the effect of perfusion of the P2Y2 agonist UTP (uridine 5′-triphosphate) on amiloride-sensitive ENaC-dependent tracheal PD. Pig tracheas were perfused with BSS between 0 to 75 min, followed by perfusion with BSS containing 0.1 mM-UTP. PD values were recorded every 1 s for 140 min. At 20, 35, 50, 65, 80, 95, 110 and 125 min 3×10⁻⁶ M-amiloride was added to the perfusing BSS or BSS+UTP for 5 min; followed by 10 min wash out with buffer. FIG. 9 is the trace of the mean data from recordings from 6 pig tracheas. Perfusion with UTP caused the baseline tracheal PD to partially depolarise, and the responses to 5 min amiloride perfusion to decrease.

### Example 6

### Amiloride Stimulates a Rapid Onset-Short Duration Increase in Ciliary Transport of Mucus Secretions in the Pig Trachea

Pig airway mucus applied to the distal end of an ex vivo pig trachea is transported by ciliary action towards the laryngeal end of the trachea. Baseline speed of ciliary transport (tracheal mucus velocity; TMV mm/min) was determined for each trachea by making at least three repeat measurements of ciliary transport before applying buffer solutions or buffer plus treatment solutions to the trachea. In FIG. 10 solutions were pipetted onto the luminal mucosal surface of tracheas at time 0 for 10 min; BSS control tracheas (open circles; n=6); 10⁻⁴ M amiloride in BSS (closed squares; n=6). At 10 min the excess solution was tipped away from the mucosal surface and measurement of TMV repeated. Further measurements of TMV were made at the times indicated in FIG. 10. TMV results at each time point are expressed as % of the baseline TMV, ciliary transport index (mean±SD). Amiloride stimulated ciliary transport of secretions from the first time point at 10 min through to 35 min after initiation of treatment of the tracheas. Maximum transport speed occurred at 25 min following initiation of treatment with amiloride. Transport speed returned to control levels by 40 min after treatment with amiloride. At 40 min, 55 min and 1 h 10 min no significant difference was observed between control tracheas treated with BSS and tracheas treated with BSS containing 10⁻⁴ M amiloride. Stimulation of ciliary transport of secretions in the pig trachea by amiloride is therefore rapid onset and of short duration (less than 1 h).

### Example 7

### Hyperosmotic Saline and 6-Amidino-2-Napthyl 4-Guanidinobenzoate each Stimulate Increased Ciliary Transport of Mucus Secretions in the Normal Pig Trachea but with Different Onset and Duration Profiles. Combination of Hyperosmotic Saline and 6-Amidino-2-Napthyl 4-Guanidinobenzoate Delivers an Enhanced Onset and Duration Profile Compared to the Single Treatments

In FIG. 11A the solutions applied to the luminal mucosal surface of tracheas at time 0 for 10 min were; BSS control tracheas (open circles; n=10); BSS containing a further 50 mM-NaCl (100 mosM-NaCl) (closed squares; n=10); BSS containing 10⁻⁵M 6-amidino-2-napthyl 4-guanidinobenzoate (closed triangles; n=10).

Hyperosmotic saline stimulated (compared to BSS controls) ciliary transport of secretions (FIG. 11A) from the first time point at 25 min through to 3 h 40 min after initiation of treatment of the tracheas. Maximum transport speed occurred at 1 h 25 min following initiation of treatment with hyperosmotic saline. The time required before maximum transport speed was reached suggests that the major stimulatory effect of hypertonic saline is exerted by actions on the physiology of the epithelial cells to influence ciliary transport. Transport speed returned to control levels by 4 h 10 min after treatment with hyperosmotic saline.

Hyperosmotic saline treatment delivered a medium-onset medium duration profile of increased ciliary transport of secretions.

6-amidino-2-napthyl 4-guanidinobenzoate also stimulated ciliary transport of secretions (FIG. 11A), but with a significantly different time course profile. Stimulation of ciliary transport was slow onset with no increase in speed (compared to BSS controls) observed at the first two time points (25 and 40 min) following treatment of the tracheas. The first significant increase in ciliary transport speed (compared to BSS controls) was observed at 1 h 10 min, and thereafter at all time points until conclusion of the study at 6 h 10 min. Maximum transport speed occurred at 4 h 10 min following initiation of treatment. The time required for initiation of increased ciliary transport and for maximum transport speed to be reached suggests that the major stimulatory effect of 6-amidino-2-napthyl 4-guanidinobenzoate is again exerted by actions on the physiology of the epithelial cells to influence ciliary transport. However the unexpected clear difference between the time course profile for 6-amidino-2-napthyl 4-guanidinobenzoate and hyperosmotic saline suggests that they each influence different cellular mechanisms and processes to achieve increased ciliary transport.

6-amidino-2-napthyl 4-guanidinobenzoate treatment delivered a slow-onset long duration profile of increased ciliary transport of secretions.

Combination of 6-amidino-2-napthyl 4-guanidinobenzoate and hypertonic saline treatment resulted in a superior ciliary transport profile compared to the single treatments (FIG. 11B). Combination delivered increased ciliary transport from the first time point (25 min) after treatment through all other time points to the end of the study at 6 h 10 min (FIG. 11B). In a second study (FIG. 12) combination of 6-amidino-2-napthyl 4-guanidinobenzoate and hypertonic saline treatment resulted in increased ciliary transport (compared to BSS controls) at all time points from 25 min through to 24 h.

Combination treatment with 6-amidino-2-napthyl 4-guanidinobenzoate and hyperosmotic saline delivered a medium-onset long duration profile of increased ciliary transport of secretions.

### Example 8

### 6-Amidino-2-Napthyl 4-Guanidinobenzoate and Combination of 6-Amidino-2-Napthyl 4-Guanidinobenzoate and Hyperosmotic Saline Stimulate Increased Ciliary Transport of Mucus Secretions in the Transgenic CF Pig Trachea

In FIG. 13A BSS containing 10⁵ M 6-amidino-2-napthyl 4-guanidinobenzoate was used to treat tracheas from transgenic CF pigs. On day 1 no increase in speed of ciliary transport of secretions was observed at the first three time points after treatment (25, 40 and 55 min). Increased ciliary transport was observed from 1 h 10 min to 4 h 40 min, with maximum increased speed at 3 h 10 min following treatment. On the second day tracheas were treated again for 10 min with BSS containing 10⁻⁵ M 6-amidino-2-napthyl 4-guanidinobenzoate at 14 h 10 min (FIG. 13A). No increase in speed of ciliary transport was seen at the first time point (25 min) after treatment on day 2; but increased ciliary transport was observed at all subsequent time points to 4 h 40 min. On day 2 maximum speed of ciliary transport was observed at 3 h 10 min following treatment.

On both days 6-amidino-2-napthyl 4-guanidinobenzoate treatment delivered a slow-onset long duration profile of increased ciliary transport of secretions. The profile in the transgenic CF pig tracheas (FIG. 13A) was as observed in normal pig tracheas (FIG. 11A).

In FIG. 13B BSS containing a combination of 10⁻⁵ M 6-amidino-2-napthyl 4-guanidinobenzoate and 50 mM-NaCl (100 mosM-NaCl) was used to treat tracheas from transgenic CF pigs. On day 1 increased speed of ciliary transport of secretions was observed from the first time points after treatment (25 min) Increased ciliary transport was observed through to 6 h 10 min, with maximum increased speed at 3 h 40 min following treatment. On the second day tracheas were treated again for 10 min with BSS containing 10⁻⁵ M 6-amidino-2-napthyl 4-guanidinobenzoate at 16 h 30 min (FIG. 13B). Increased speed of ciliary transport was again seen at the first time point (25 min) after treatment on day 2; and increased ciliary transport was observed at all subsequent time points to 4 h 40 min. On day 2 maximum speed of ciliary transport was observed at 2 h 40 min following treatment.

On both days combination of 6-amidino-2-napthyl 4-guanidinobenzoate and hyperosmotic saline treatment delivered a medium/rapid-onset long duration profile of increased ciliary transport of secretions. The profile in the transgenic CF pig tracheas (FIG. 13B) was as observed in normal pig tracheas (FIGS. 11B & 12).

### Example 9

### Hyperosmotic Mannitol Stimulates a Medium Onset-Medium Duration Increase in Ciliary Transport of Mucus Secretions in the Pig Trachea

In FIG. 14A the solutions applied to the luminal mucosal surface of tracheas at time 0 for 10 min were; BSS control tracheas (open circles; n=7); BSS containing 100 mosM-mannitol (closed squares; n=7); BSS containing 10⁻⁵M 6-amidino-2-napthyl 4-guanidinobenzoate and 100mosM-mannitol (closed circles; n=7).

Hyperosmotic mannitol stimulated (compared to BSS controls) ciliary transport of secretions (FIG. 14A) from the first time point at 25 min through to 2 h 25 min after initiation of treatment of the tracheas. Maximum transport speed occurred at 1 h 40 min following initiation of treatment with hyperosmotic mannitol. The time required before maximum transport speed was reached suggests that the major stimulatory effect of hyperosmotic mannitol is exerted by actions on the physiology of the epithelial cells to influence ciliary transport. Transport speed returned to control levels by 2 h 40 min after treatment with hyperosmotic mannitol.

Hyperosmotic mannitol treatment delivered a medium-onset medium duration profile of increased ciliary transport of secretions.

Combination of 6-amidino-2-napthyl 4-guanidinobenzoate and hyperosmotic mannitol treatment resulted in a superior ciliary transport profile compared to treatment with only hyperosmotic mannitol (FIG. 14A). Combination delivered increased ciliary transport from the first time point (25 min) after treatment through all other time points to the end of the study at 3 h 10 min (FIG. 14A).

Combination treatment with 6-amidino-2-napthyl 4-guanidinobenzoate and hyperosmotic mannitol delivered a medium-onset long duration profile of increased ciliary transport of secretions.

### Example 10

### Hyperosmotic Sodium Gluconate Stimulates a Medium Onset-Medium Duration Increase in Ciliary Transport of Mucus Secretions in the Pig Trachea

In FIG. 14B the solutions applied to the luminal mucosal surface of tracheas at time 0 for 10 min were; BSS control tracheas (open circles; n=5); BSS containing 100 mosM-sodium gluconate (closed squares; n=6); BSS containing 10⁻⁵M 6-amidino-2-napthyl 4-guanidinobenzoate and 100 mosM-sodium gluconate (closed circles; n=6).

Hyperosmotic sodium gluconate stimulated (compared to BSS controls) ciliary transport of secretions (FIG. 14B) from the first time point at 25 min through to 3 h 10 min after initiation of treatment of the tracheas. Maximum transport speed occurred at 1 h 40 min following initiation of treatment with hyperosmotic sodium gluconate. The time required before maximum transport speed was reached suggests that the major stimulatory effect of hyperosmotic sodium gluconate is exerted by actions on the physiology of the epithelial cells to influence ciliary transport.

Hyperosmotic sodium gluconate treatment delivered a medium-onset medium duration profile of increased ciliary transport of secretions.

Combination of 6-amidino-2-napthyl 4-guanidinobenzoate and hyperosmotic sodium gluconate treatment resulted in a superior ciliary transport profile compared to treatment with only hyperosmotic sodium gluconate (FIG. 14B). Combination delivered increased ciliary transport from the first time point (25 min) after treatment through all other time points to the end of the study at 3 h 10 min (FIG. 14B).

Combination treatment with 6-amidino-2-napthyl 4-guanidinobenzoate and hyperosmotic sodium gluconate delivered a medium-onset long duration profile of increased ciliary transport of secretions.

### Example 11

### UTP Stimulates a Medium Onset-Medium Duration Increase in Ciliary Transport of Mucus Secretions in the Pig Trachea

In FIG. 14C the solutions applied to the luminal mucosal surface of tracheas at time 0 for 10 min were; BSS control tracheas (open circles; n=5); BSS containing 0.1 mM UTP (closed squares; n=10); BSS containing 10⁻⁵M 6-amidino-2-napthyl 4-guanidinobenzoate and 0.1 mM UTP (closed circles; n=10).

UTP stimulated (compared to BSS controls) ciliary transport of secretions (FIG. 14C) from the first time point at 25 min through to 1 h 40 min after initiation of treatment of the tracheas. Maximum transport speed occurred at 55 min following initiation of treatment with UTP. The time required before maximum transport speed was reached suggests that the major stimulatory effect of UTP is exerted by actions on the physiology of the epithelial cells to influence ciliary transport.

UTP treatment delivered a medium-onset medium duration profile of increased ciliary transport of secretions.

Combination of 6-amidino-2-napthyl 4-guanidinobenzoate and UTP treatment resulted in a superior ciliary transport profile compared to treatment with only UTP (FIG. 14C). Combination delivered increased ciliary transport from the first time point (25 min) after treatment through all other time points to the end of the study at 3 h 10 min (FIG. 14C).

Combination treatment with 6-amidino-2-napthyl 4-guanidinobenzoate and UTP delivered a medium-onset long duration profile of increased ciliary transport of secretions.

### Example 12

### Dose-Related Stimulation of Ciliary Transport of Mucus Secretions in the Pig Trachea by 6-Amidino-2-Napthyl 4-Guanidinobenzoate

Pig tracheas were treated with 6-amidino-2-napthyl 4-guanidinobenzoate in the dose range 10⁻⁹ M to 10⁻⁵ M, and ciliary transport of mucus secretions was measured for 4 h. Peak increase in ciliary transport speed occurred at 3 h 40 min following treatment. FIG. 15 shows the dose-response profile at 3 h 40 min. Increased ciliary transport occurred over the dose range 10⁻⁸ M to 10⁻⁵ M, maximum increase in ciliary transport occurred at 10⁻⁶ M 6-amidino-2-napthyl 4-guanidinobenzoate.

### REFERENCES

Bilton D, Bellon G, Charlton B, Cooper P, De Boeck K, Flume P A, Fox H G, Gallagher C G, Geller D E, Haarman E G, Hebestreit H U, Kolbe J, Lapey A, Robinson P, Wu J, Zuckerman J B, Aitken M L
Pooled analysis of two large randomised phase III inhaled mannitol studies in cystic fibrosis.
J Cyst Fibros. 2013; 12: 367-76
Bridges R J, Newton B B, Pilewski J M, Devor D C, Poll C T. & Hall R L.
Na+ transport in normal and CF human bronchial epithelial cells is inhibited by BAY 39-9437.
Am J Physiol Lung Cell Mol Physiol. 2001; 281: L16-23.
Danahay H, Atherton H, Jones G, Bridges R J. & Poll C T.
Interleukin-13 induces a hypersecretory ion transport phenotype in human bronchial epithelial cells.
Am J Physiol Lung Cell Mol Physiol. 2002; 282: L226-236.
Elkins M R, Robinson M, Rose B R, Harbour C, Moriarty C P, Marks G B, Belousova E G, Xuan W & Bye P T P
A Controlled Trial of Long-Term Inhaled Hypertonic Saline in Patients with Cystic Fibrosis.
N Engl J Med 2006; 354: 229-240
Hitomi Y, Ikari N, Fujii S
Inhibitory effect of a new synthetic protease inhibitor (FUT-175) on the coagulation system. Haemostasis. 1985;15: 164-8
Iwaki M, Oda M, Ozeki M, Ino Y, Suzuki K, Koshiyama Y, Motoyoshi A, Ogihara M, Suzuki S, Fujita M, et al.
[Pharmacological studies of FUT-175, nafamstat mesilate. III. Anti-inflammatory activities of FUT-175].
Nippon Yakurigaku Zasshi. 1984;84: 373-84.
Iwashita K, Kitamura K, Narikiyo T, Adachi M, Shiraishi N, Miyoshi T, Nagano J, Tuyen D G, Nonoguchi H. & Tomita K.
Inhibition of prostasin secretion by serine protease inhibitors in the kidney.
J Am Soc Nephrol. 2003;14: 11-16.
JP57053454
Klymiuk N, Mundhenk L, Kraehe K, Wuensch A, Plog S, Emrich D, Langenmayer M C, Stehr M, Holzinger A, Kröner C, Richter A, Kessler B, Kurome M, Eddicks M, Nagashima H, Heinritzi K, Gruber A D & Wolf E.
Sequential targeting of CFTR by BAC vectors generates a novel pig model of cystic fibrosis.
J Mol Med. 2012; 90: 597-608

Min Y G, Lee K S, Yun J B, Rhee C S, Rhyoo C, Koh Y Y, Yi W J. & Park K S.
Hypertonic saline decreases ciliary movement in human nasal epithelium in vitro.
Otolaryngol Head Neck Surg. 2001; 124: 313-316.
Muto, S, Imai M. & Asano Y.
Effect of nafamostat mesilate on Na+ and K+ transport properties in the rabbit cortical collecting duct.
Br. J. Pharmacol. 1993; 109: 673-678.
Muto S, Imai M. & Asano Y.
Mechanisms of the hyperkalaemia caused by nafamostat mesilate: effects of its two metabolites on Na+ and K+ transport properties in the rabbit cortical collecting duct. Br. J. Pharmacol. 1994; 111: 173-178.
Narikiyo T, Kitamura K, Adachi M, Miyoshi T, Iwashita K, Shiraishi N, Nonoguchi H, Chen L M, Chai K X, Chao J. & Tomita K.
Regulation of prostasin by aldosterone in the kidney.
J Clin Invest. 2002; 109: 401-408.
Rasgado-Flores H, Mandava V K. & Bridges R J.
Effect of osmolarity and amiloride on sodium transport in cystic fibrosis and non-cystic fibrosis human bronchial epithelial cells.
Pediatric Pulmonol. 2009; S32; 156-157
Rasgado-Flores H, Mandava V K, Siman H, Van Driessche W, Pilewski J M, Randell S H. & Bridges R J.
Effect of apical hyperosmotic sodium challenge and amiloride on sodium transport in human bronchial epithelial cells from cystic fibrosis donors.
Am J Physiol Cell Physiol. 2013; 305: C1114-1122.
Rossier B C.
The epithelial sodium channel: activation by membrane-bound serine proteases.
Proc Am Thorac Soc. 2004; 1: 4-9.
Standaert T A, Boitano L, Emerson J, Milgram L J, Konstan M W, Hunter J, Berclaz P Y, Brass L, Zeitlin P L, Hammond K, Davies Z, Foy C, Noone P G, Knowles M R.
Standardized procedure for measurement of nasal potential difference: an outcome measure in multicenter cystic fibrosis clinical trials.
Pediatr Pulmonol. 2004;37(5):385-92.
Tong Z, Illek B, Bhagwandin V J, Verghese G M. & Caughey G H.
Prostasin, a membrane-anchored serine peptidase, regulates sodium currents in JME/CF15 cells, a cystic fibrosis airway epithelial cell line.
Am J Physiol Lung Cell Mol Physiol. 2004; 287: L928-935.
U.S. Pat. No. 4,454,338
Vallet V, Chraibi A, Gaeggeler H P, Horisberger J D. & Rossier B C.
An epithelial serine protease activates the amiloride-sensitive sodium channel.
Nature. 1997; 389: 607-610.
WO 2008/090366,
Wills P J, Hall R L, Chan W. & Cole P J.
Sodium chloride increases the ciliary transportability of cystic fibrosis and bronchiectasis sputum on the mucus-depleted bovine trachea
J.Clin.Invest. 1997; 99: 9-13.

1. An amidino compound of the general formula: 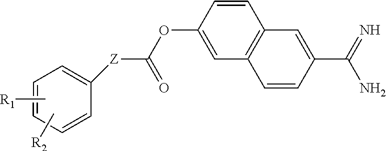
and a hyperosmotic agent or a purinergic agonist for use in a method of treating respiratory disease wherein;
Z represents —(CH₂)a-, 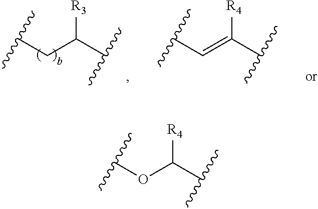
wherein a is 0, 1, 2 or 3, b is 0,1 or 2, R₃ is a straight or branched chain alkyl group of 1 to 4 carbon atoms or a cycloalkyl group of 3 to 6 carbon atoms, R₄ is a hydrogen atom or a straight or branched chain alkyl group of 1 to 4 carbon atoms;
R₁ and R₂, may be the same or different and represent each a hydrogen atom, a straight or branched chain alkyl group of 1 to 4 carbon atoms, —O—R₅, —S—R₅, —COOR₅, —COR₆, —O—COR₇, —NHCOR₇, 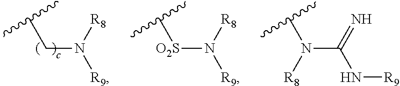
NO₂, CN, halogen, CF₃, methylenedioxy, or 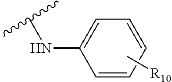
wherein c is 0, 1 or 2; R₅ is a hydrogen atom, linear or branched chain alkyl group of 1 to 4 carbon atoms, or benzyl group; R₆ is a hydrogen atom or straight or branched chain alkyl group of 1 to 4 carbon atoms; R₇ is a straight or branched chain alkyl group of 1 to 4 carbon atoms; R₈ and R₉, which may be the same or different, are each a hydrogen atom, straight or branched chain alkyl group of 1 to 4 carbon atoms, or amino radical protecting group; and R₁₀ is a hydrogen atom, methyl or CF₃.

2. The amiditio compound and hyperosmotic agent or purinergic agonist of claim 1 wherein Z represents a covalent bond.

3. The amidino compound and hyperosmotic agent or purinergic agonist of claim 1 or claim 2 wherein R₁ or R₂ represent hydrogen or a straight or branched alkyl group having from 1 to 4 carbon atoms.

4. The amidino compound and hyperosmotic agent or purinergic agonist of any one of claims 1 to 3 wherein R₁ is hydrogen and R₂ is 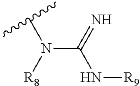
wherein R₈ and R₉ are hydrogen.

5. The amidino compound and hyperosmotic agent or purinergic agonist of any preceding claim, wherein the amidino compound is in the form of a pharmaceutically acceptable salt or ester.

6. The amidino compound and hyperosmotic agent or purinergic agonist of any preceding claim, wherein the amidino compound is 6-amidino-2-napthyl 4-guanidinobenzoate.

7. The amidino compound and hyperosmotic agent or purinergic agonist of claim 6, wherein the 6-amidino-2-napthyl 4-guanidinobenzoate is in the form of 6-amidino-2-napthyl 4-guanidinobenzoate dihydrochloride or 6-amidino-2-napthyl 4-guanidinobenzoate mesylate.

8. The amidino compound and hyperosmotic agent or purinergic agonist of any of the preceding claims comprising a hyperosmotic agent.

9. The amidino compound and hyperosmotic agent of claim 8, wherein the hyperosmotic agent increases the osmolarity of the recipient's lungs to between 350 mosM to 600 mosM.

10. The amidino compound and hyperosmotic agent of claim 9, wherein the increase in osmolarity in the recipient's lungs is achieved by inhalation as a nebulized aerosol of 0.5 ml to 20 ml of a 600 mosM to 3000 mosM solution.

11. The amidino compound and hyperosmotic agent of claim 9, wherein the increase in osmolarity in the recipient's lungs is achieved by inhalation as a dry powder formulation of 0.5 to 3 mosmoles of a hyperosmotic agent.

12. The amidino compound and hyperosmotic agent of claim 8, wherein the hyperosmotic agent is hypertonic saline.

13. The amidino compound and hyperosmotic agent of claim 12 wherein the hypertonic saline increases the osmolarity of the recipient's lungs to between 350 mosM to 600 mosM.

14. The amidino compound and hyperosmotic agent of claim 13, wherein the increase in osmolarity in the recipient's lungs is achieved by inhalation as a nebulized aerosol of 0.5 ml to 20 ml of 600 mosM to 3000 mosM hypertonic saline.

15. The amidino compound and hyperosmotic agent of claim 8, wherein the hyperosmotic agent is mannitol.

16. The amidino compound and hyperosmotic agent of claim 15, wherein the mannitol increases the osmolarity of the recipient's lungs to between 350 mosM to 600 mosM.

17. The amidino compound and hyperosmotic agent of claim 16, wherein the increase in osmolarity in the recipient's lungs is achieved by inhalation as a nebulized aerosol of 0.5 ml to 20 ml of 600 mosM to 3000 mosM mannitol.

18. The amidino compound and hyperosmotic agent of claim 16, wherein the increase in osmolarity in the recipient's lungs is achieved by inhalation as a dry powder formulation of 0.5 to 3 mosmoles of mannitol.

19. The amidino compound and hyperosmotic agent agonist of claim 8, wherein the hyperosmotic agent is sodium gluconate.

20. The amidino compound and hyperosmotic agent of claim 19, wherein the sodium gluconate increases the osmolarity of the recipient's lungs to between 350 mosM to 600 mosM.

21. The amidino compound and hyperosmotic agent of claim 20, wherein the increase in osmolarity in the recipient's lungs is achieved by inhalation as a nebulized aerosol of 0.5 ml to 20 ml of 600 mosM to 3000 mosM sodium gluconate.

22. The amidino compound and hyperosmotic agent of claim 20, wherein the increase in osmolarity in the recipient's lungs is achieved by inhalation as a dry powder formulation of 0.5 to 3 mosmoles of sodium gluconate.

23. The amidino compound and hyperosmotic agent or purinergic agonist of any one of claims 1-7 comprising a purinergic agonist.

24. The amidino compound and purinergic agonist of claim 23 wherein the purinergic agonist is uridine-5′-triphosphate (UTP), P¹,P⁴-bis(5′-uridyl) tetraphosphate tetrasodium salt (Diquafosol), or 2′-deoxycytidine(5′) tetraphospho (5′) uridine tetrasodium salt (Denufosol).

25. The amidino compound and purinergic agonist of claim 23 or claim 24, wherein the purinergic agonist is administered by inhalation.

26. The amidino compound and purinergic agonist of any one of claims 23-25, wherein up to 60 mg of the purinergic agonist is administered.

27. The amidino compound and hyperosmotic agent or purinergic agonist of any one of the preceding claims wherein the amidino compound and hyperosmotic agent or purinergic agonist are for combined, separate or sequential administration.

28. The amidino compound and hyperosmotic agent or purinergic agonist of any one of the preceding claim, wherein the amidino compound and/or the hyperosmotic agent or purinergic agonist further comprises one or more pharmaceutically acceptable carriers or diluents.

29. The amidino compound and hyperosmotic agent or purinergic agonist of any one of the preceding claims, wherein the amidino compound and/or the hyperosmotic agent or purinergic agonist is in aqueous solution.

30. The amidino compound and hyperosmotic agent or purinergic agonist of any one of the preceding claims wherein the amidino compound and/or the hyperosmotic agent or purinergic agonist also comprises one or more other active agents selected from the group of antibiotics, vaccines, decongestants (nasal or bronchial), rhDNase, non-steroidal antiinflamatory agents (NSAIDs), steroids, antiviral agents, elastase inhibitors, exofacial sodium channel blocking agents, gene therapy agents, chloride channel activators and bronchodilators.

31. The amidino compound and hyperosmotic agent or purinergic agonist of any one of the preceding claims, wherein the respiratory disease is characterised by poor mucociliary clearance or mucostasis.

32. The amidino compound and hyperosmotic agent or purinergic agonist of any one of the preceding claims, wherein the respiratory disease is selected from the group comprising cystic fibrosis, bronchiectasis, chronic bronchitis, chronic obstructive pulmonary disease (COPD), rhino-sinusitis and otitis media.

33. The amidino compound and hyperosmotic agent or purinergic agonist of claim 32, wherein the respiratory disease is cystic fibrosis.

34. The amidino compound and hyperosmotic agent or purinergic agonist of any one of the preceding claims, wherein the amidino compound and/or the hyperosmotic agent or purinergic agonist is formulated for administration to the respiratory system by the pulmonary route.

35. The amidino compound and hyperosmotic agent or purinergic agonist of claim 34, wherein the amidino compound and/or the hyperosmotic agent or purinergic agonist is formulated for administration by any one of intratracheal installation, intratracheal delivery of liposomes, insufflation, nebulization, dry powder inhalation and aerosol inhalation.

36. The amidino compound and hyperosmotic agent or purinergic agonist of claim 35, wherein the amidino compound and/or the hyperosmotic agent or purinergic agonist is formulated for administration by dry powder inhalation or aerosol inhalation, together with a propellant selected from the group comprising hydrofluroalkanes, chlorofluorocarbons, propane, nitrogen, or a mixture thereof.

37. The amidino compound and hyperosmotic agent or purinergic agonist of any one of the preceding claims, wherein the amidino compound and/or the hyperosmotic agent or purinergic agonist is formulated for the treatment of the nasal epithelium, para-nasal sinuses, the Eustachian tube or middle ear, said medicaments being in the form of solutions for delivery as drops, by pipette or by syringe, or by aerosol to the nasal epithelium, para-nasal sinuses, the Eustachian tube or middle ear, for the irrigation of the para-nasal sinuses, or for delivery as solution directly applied to the middle ear via the external auditory meatus and canal.

38. A composition comprising an amidino compound of the general formula: 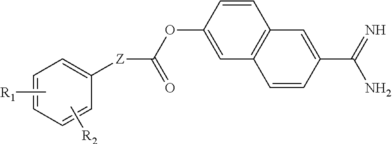
and a hyperosmotic agent or a purinergic agonist wherein;
Z represents —(CH₂)a-, 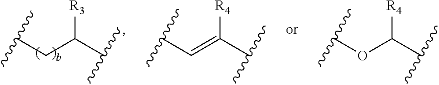
wherein a is 0, 1, 2 or 3, b is 0,1 or 2, R₃ is a straight or branched chain alkyl group of 1 to 4 carbon atoms or a cycloalkyl group of 3 to 6 carbon atoms, R₄ is a hydrogen atom or a straight or branched chain alkyl group of 1 to 4 carbon atoms;
R₁ and R₂, may be the same or different and represent each a hydrogen atom, a straight or branched chain alkyl group of 1 to 4 carbon atoms, —O—R₅, —S—R₅, —COOR₅, —COR₆, —O—COR₇, —NHCOR₇, 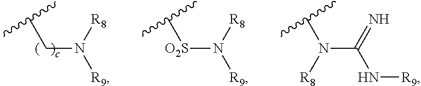
NO₂, CN, halogen, CF₃, methylenedioxy, or 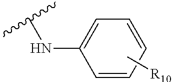
wherein c is 0, 1 or 2; R₅ is a hydrogen atom, linear or branched chain alkyl group of 1 to 4 carbon atoms, or benzyl group; R₆ is a hydrogen atom or straight or branched chain alkyl group of 1 to 4 carbon atoms; R₇ is a straight or branched chain alkyl group of 1 to 4 carbon atoms; R₈ and R₉, which may be the same or different, are each a hydrogen atom, straight or branched chain alkyl group of 1 to 4 carbon atoms, or amino radical protecting group; and R₁₀ is a hydrogen atom, methyl or CF₃.

39. The composition of claim 38 wherein Z represents a covalent bond.

40. The composition of claim 38 or claim 39 wherein R₁ or R₂ represent hydrogen or a straight or branched alkyl group having from 1 to 4 carbon atoms.

41. The composition of any one of claims 38 to 40 wherein R₁ is hydrogen and R₂ is 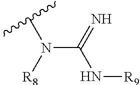
wherein R₈ and R₉ are hydrogen.

42. The composition of any one of claims 38-41, wherein the amidino compound is in the form of a pharmaceutically acceptable salt or ester.

43. The composition of any one of claims 38-42, wherein the amidino compound is 6-amidino-2-napthyl 4-guanidinobenzoate.

44. The composition of claim 43, wherein the 6-amidino-2-napthyl 4-guanidinobenzoate is in the form of 6-amidino-2-napthyl 4-guanidinobenzoate dihydrochloride or 6-amidino-2-napthyl 4-guanidinobenzoate mesylate.

45. The composition of any one of claims 38-44 comprising a hyperosmotic agent.

46. The composition of claim 45, wherein the hyperosmotic agent increases the osmolarity of the recipient's lungs to between 350 mosM to 600 mosM.

47. The composition of claim 46, wherein the increase in osmolarity in the recipient's lungs is achieved by inhalation as a nebulized aerosol of 0.5 ml to 20 ml of 600 mosM to 3000 mosM solution.

48. The composition of claim 46, wherein the increase in osmolarity in the recipient's lungs is achieved by inhalation as a dry powder formulation of 0.5 to 3 mosmoles of a hyperosmotic agent.

49. The composition of claim 45, wherein the hyperosmotic agent is hypertonic saline.

50. The composition of claim 49 wherein the hypertonic saline increases the osmolarity of the recipient's lungs to between 350 mosM to 600 mosM.

51. The composition of claim 50, wherein the increase in osmolarity in the recipient's lungs is achieved by inhalation as a nebulized aerosol of 0.5 ml to 20 ml of 600 mosM to 3000 mosM hypertonic saline.

52. The composition of claim 45, wherein the hyperosmotic agent is mannitol.

53. The composition of claim 52, wherein the mannitol increases the osmolarity of the recipient's lungs to between 350 mosM to 600 mosM.

54. The composition of claim 53, wherein the increase in osmolarity in the recipient's lungs is achieved by inhalation as a nebulized aerosol of 0.5 ml to 20 ml of 600 mosM to 3000 mosM mannitol.

55. The composition of claim 53, wherein the increase in osmolarity in the recipient's lungs is achieved by inhalation as a dry powder formulation of 0.5 to 3 mosmoles of mannitol.

56. The composition of claim 45, wherein the hyperosmotic agent is sodium gluconate.

57. The composition of claim 56, wherein the sodium gluconate increases the osmolarity of the recipient's lungs to between 350 mosM to 600 mosM.

58. The composition of claim 57, wherein the increase in osmolarity in the recipient's lungs is achieved by inhalation as a nebulized aerosol of 0.5 ml to 20 ml of 600 mosM to 3000 mosM sodium gluconate.

59. The composition of claim 57, wherein the increase in osmolarity in the recipient's lungs is achieved by inhalation as a dry powder formulation of 0.5 to 3 mosmoles of sodium gluconate.

60. The composition of any one of claims 38-44 comprising a purinergic agonist.

61. The composition of claim 60 wherein the purinergic agonist is uridine-5′-triphosphate (UTP), P¹,P⁴-bis(5′-uridyl) tetraphosphate tetrasodium salt (Diquafosol), or 2′-deoxycytidine(5′) tetraphospho (5′) uridine tetrasodium salt (Denufosol).

62. The composition of claim 60 or claim 61, wherein the purinergic agonist is administered by inhalation.

63. The composition of any one of claims 60-62, wherein up to 60mg of the purinergic agonist is administered.

64. The composition of any one of claims 38-63 further comprising one or more pharmaceutically acceptable carriers or diluents.

65. The composition of any one of claims 38-64 which is in aqueous solution.

66. The composition of any one of claims 38-65 further comprising one or more other active agents selected from the group of antibiotics, vaccines, decongestants (nasal or bronchial), rhDNase, non-steroidal antiinflamatory agents (NSAIDs), steroids, antiviral agents, elastase inhibitors, exofacial sodium channel blocking agents, gene therapy agents, chloride channel activators and bronchodilators.

67. The composition of any one of claims 38-66 for use in a method of treating respiratory disease.

68. The composition of claim 67, wherein the respiratory disease is characterised by poor mucociliary clearance or mucostasis.

69. The composition of claim 67 or 68, wherein the respiratory disease is selected from the group comprising cystic fibrosis, bronchiectasis, chronic bronchitis, chronic obstructive pulmonary disease (COPD), rhino-sinusitis and otitis media.

70. The compound of claim 69, wherein the respiratory disease is cystic fibrosis.

71. The composition of any one of claims 67-70, wherein the composition is formulated for administration to the respiratory system by the pulmonary route.

72. The composition of claim 71, wherein the composition is formulated for administration by any one of intratracheal installation, intratracheal delivery of liposomes, insufflation, nebulization, dry powder inhalation and aerosol inhalation.

73. The composition of claim 72, wherein the composition is formulated for administration by dry powder inhalation or aerosol inhalation, together with a propellant selected from the group comprising hydrofluroalkanes, chlorofluorocarbons, propane, nitrogen, or a mixture thereof.

74. The composition of any one of claims 67-73, wherein the composition is formulated for the treatment of the nasal epithelium, para-nasal sinuses, the Eustachian tube or middle ear, said medicaments being in the form of solutions for delivery as drops, by pipette or by syringe, or by aerosol to the nasal epithelium, para-nasal sinuses, the Eustachian tube or middle ear, for the irrigation of the para-nasal sinuses, or for delivery as solution directly applied to the middle ear via the external auditory meatus and canal.

75. An inhalation device loaded with the pharmaceutical composition of any one of claims 38-74.

76. The inhalation device of claim 75, wherein the device is a dry powder inhaler, metered dose inhaler, jet nebulizer or ultrasonic nebulizer.

77. A method of treating a respiratory disease, comprising administering a therapeutically effective amount of an amidino compound of the general formula: 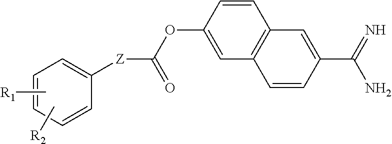
and a hyperosmotic agent or a purinergic agonist, wherein;
Z represents —(CH₂)a-, 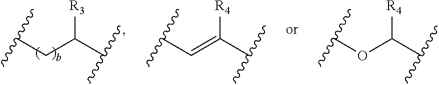
wherein a is 0, 1, 2 or 3, b is 0,1 or 2, R₃ is a straight or branched chain alkyl group of 1 to 4 carbon atoms or a cycloalkyl group of 3 to 6 carbon atoms, R₄ is a hydrogen atom or a straight or branched chain alkyl group of 1 to 4 carbon atoms;
R₁ and R₂, may be the same or different and represent each a hydrogen atom, a straight or branched chain alkyl group of 1 to 4 carbon atoms, —O—R₅, —S—R₅, —COOR₅, —COR₆, —O—COR₇, —NHCOR₇, 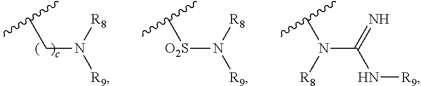
NO₂, CN, halogen, CF₃, methylenedioxy, or 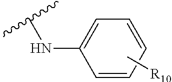
wherein c is 0, 1 or 2; R₅ is a hydrogen atom, linear or branched chain alkyl group of 1 to 4 carbon atoms, or benzyl group; R₆ is a hydrogen atom or straight or branched chain alkyl group of 1 to 4 carbon atoms; R₇ is a straight or branched chain alkyl group of 1 to 4 carbon atoms; R₈ and R₉, which may be the same or different, are each a hydrogen atom, straight or branched chain alkyl group of 1 to 4 carbon atoms, or amino radical protecting group; and R₁₀ is a hydrogen atom, methyl or CF_{3;}
to a patient in need thereof.

78. The method claim 77 wherein Z represents a covalent bond.

79. The method of claim 77 or claim 78 wherein R₁ or R₂ represent hydrogen or a straight or branched alkyl group having from 1 to 4 carbon atoms.

80. The method of any one of claims 77 to 79 wherein R₁ is hydrogen and R₂ is 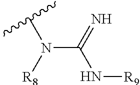
wherein R₈ and R₉ are hydrogen.

81. The method of any one of claims 77-80, wherein the amidino compound is in the form of a pharmaceutically acceptable salt or ester.

82. The method of any one of claims 77-81, wherein the amidino compound is 6-amidino-2-napthyl 4-guanidinobenzoate.

83. The method of claim 82, wherein the 6-amidino-2-napthyl 4-guanidinobenzoate is in the form of 6-amidino-2-napthyl 4-guanidinobenzoate dihydrochloride or 6-amidino-2-napthyl 4-guanidinobenzoate mesylate.

84. The method of any one of claims 77-83 comprising administering a hyperosmotic agent.

85. The method of claim 84, wherein the hyperosmotic agent increases the osmolarity of the recipient's lungs to between 350 mosM to 600 mosM.

86. The method of claim 85, wherein the increase in osmolarity in the recipient's lungs is achieved by inhalation as a nebulized aerosol of 0.5 ml to 20 ml of 600 mosM to 3000 mosM solution.

87. The method of claim 86, wherein the increase in osmolarity in the recipient's lungs is achieved by inhalation as a dry powder formulation of 0.5 to 3 mosmoles of a hyperosmotic agent.

88. The method of claim 84, wherein the hyperosmotic agent is hypertonic saline.

89. The method of claim 88, wherein the hypertonic saline increases the osmolarity of the recipient's lungs to between 350 mosM to 600 mosM.

90. The method of claim 89, wherein the increase in osmolarity in the recipient's lungs is achieved by inhalation as a nebulized aerosol of 0.5 ml to 20 ml of 600 mosM to 3000 mosM hypertonic saline.

91. The method of claim 84, wherein the hyperosmotic agent is mannitol.

92. The method of claim 91, wherein the mannitol increases the osmolarity of the recipient's lungs to between 350 mosM to 600 mosM.

93. The method of claim 92, wherein the increase in osmolarity in the recipient's lungs is achieved by inhalation as a nebulized aerosol of 0.5 ml to 20 ml of 600 mosM to 3000 mosM mannitol.

94. The method of claim 92, wherein the increase in osmolarity in the recipient's lungs is achieved by inhalation as a dry powder formulation of 0.5 to 3 mosmoles of mannitol.

95. The method of claim 84, wherein the hyperosmotic agent is sodium gluconate.

96. The method of claim 95, wherein the sodium gluconate increases the osmolarity of the recipient's lungs to between 350 mosM to 600 mosM.

97. The method of claim 96, wherein the increase in osmolarity in the recipient's lungs is achieved by inhalation as a nebulized aerosol of 0.5 ml to 20 ml of 600 mosM to 3000 mosM sodium gluconate.

98. The method of claim 96, wherein the increase in osmolarity in the recipient's lungs is achieved by inhalation as a dry powder formulation of 0.5 to 3 mosmoles of sodium gluconate.

99. The method of any one of claims 77-83 comprising administering a purinergic agonist.

100. The method of claim 99, wherein the purinergic agonist is uridine-5′-triphosphate (UTP), P¹,P⁴-bis(5′-uridyl) tetraphosphate tetrasodium salt (Diquafosol), or 2′-deoxycytidine(5′) tetraphospho (5′) uridine tetrasodium salt (Denufosol).

101. The method of claim 99 or claim 100, wherein the purinergic agonist is administered by inhalation.

102. The method of any one of claims 99-101, wherein up to 60mg of the purinergic agonist is administered.

103. The method of any one of claims 77-102, wherein the amidino compound and hyperosmotic agent or purinergic agonist are for combined, separate or sequential administration.

104. The method of any one of claims 77-103, wherein the amidino compound and/or the hyperosmotic agent or purinergic agonist is administered together with one or more pharmaceutically acceptable carriers or diluents.

105. The method of any one of claims 77-104, wherein the amidino compound and/or the hyperosmotic agent or purinergic agonist is administered as an aqueous solution.

106. The method of any one of claims 77-105, wherein the amidino compound and/or the hyperosmotic agent or purinergic agonist is administered in combination with one or more other active agents selected from the group of antibiotics, vaccines, decongestants (nasal or bronchial), rhDNase, non-steroidal antiinflamatory agents (NSAIDs), steroids, antiviral agents, elastase inhibitors, exofacial sodium channel blocking agents, gene therapy agents, chloride channel activators and bronchodilators.

107. The method of any one of claims 77-106, wherein the respiratory disease is characterised by poor mucociliary clearance or mucostasis.

108. The method of any one of claims 77-107, wherein the respiratory disease is selected from the group comprising cystic fibrosis, bronchiectasis, chronic bronchitis, chronic obstructive pulmonary disease (COPD), rhino-sinusitis and otitis media.

109. The method of claim 108, wherein the respiratory disease is cystic fibrosis.

110. The method of any one of claims 77-109, wherein the administration is to the respiratory system by the pulmonary route.

111. The method of claim 110, wherein the administration is by any one of intratracheal installation, intratracheal delivery of liposomes, insufflation, nebulization, dry powder inhalation and aerosol inhalation.

112. The method of claim 111, wherein the administration is by dry powder inhalation or aerosol inhalation, together with a propellant selected from the group comprising hydrofluroalkanes, chlorofluorocarbons, propane, nitrogen, or a mixture thereof.

113. The method of any one of claims 77-112, wherein the administration is as drops, by pipette or by syringe, or by aerosol to the nasal epithelium, para-nasal sinuses, the Eustachian tube or middle ear, for irrigation of the para-nasal sinuses, or by solution directly applied to the middle ear via the external auditory meatus and canal.

114. An Amiloride-sensitive sodium channel (ENaC) regulator for use in a method of increasing ciliary transport of mucus secretions.

115. The Amiloride-sensitive sodium channel (ENaC) regulator of claim 114, wherein the Amiloride-sensitive sodium channel (ENaC) regulator is selected from the group comprising Amiloride and other exofacial sodium channel blockers; hypertonic saline, hyperosmotic mannitol, hyperosmotic sodium gluconate, and other hyperosmotic treatments; amidino compounds and Kunitz-type serine protease inhibitors.

116. The Amiloride-sensitive sodium channel (ENaC) regulator of claim 115, wherein the Amiloride-sensitive sodium channel (ENaC) regulator is an amidino compound of the general formula: 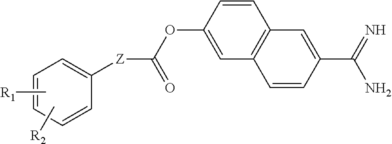
Wherein Z represents —(CH₂)a-, 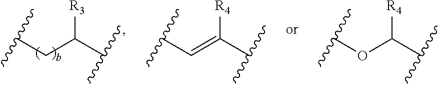
wherein a is 0, 1, 2 or 3, b is 0,1 or 2, R₃ is a straight or branched chain alkyl group of 1 to 4 carbon atoms or a cycloalkyl group of 3 to 6 carbon atoms, R₄ is a hydrogen atom or a straight or branched chain alkyl group of 1 to 4 carbon atoms;
R₁ and R₂, may be the same or different and represent each a hydrogen atom, a straight or branched chain alkyl group of 1 to 4 carbon atoms, —O—R₅, —S—R₅, —COOR₅, —COR₆, —O—COR₇, —NHCOR₇, 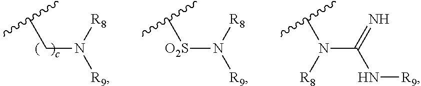
NO₂, CN, halogen, CF₃, methylenedioxy, or 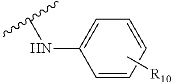
wherein c is 0, 1 or 2; R₅ is a hydrogen atom, linear or branched chain alkyl group of 1 to 4 carbon atoms, or benzyl group; R₆ is a hydrogen atom or straight or branched chain alkyl group of 1 to 4 carbon atoms; R₇ is a straight or branched chain alkyl group of 1 to 4 carbon atoms; R₈ and R₉, which may be the same or different, are each a hydrogen atom, straight or branched chain alkyl group of 1 to 4 carbon atoms, or amino radical protecting group; and R₁₀ is a hydrogen atom, methyl or CF₃.

117. The Amiloride-sensitive sodium channel (ENaC) regulator of claim 116, wherein Z represents a covalent bond.

118. The Amiloride-sensitive sodium channel (ENaC) regulator of claim 116 or claim 117, wherein R₁ or R₂ represent hydrogen or a straight or branched alkyl group having from 1 to 4 carbon atoms.

119. The Amiloride-sensitive sodium channel (ENaC) regulator of any one of claims 116 to 118 wherein. R₁ is hydrogen and R₂ is 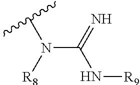
wherein R₈ and R₉ are hydrogen.

120. The Amiloride-sensitive sodium channel (ENaC) regulator of any one of claims 116 to 119, wherein the amidino compound is in the form of a pharmaceutically acceptable salt or ester.

121. The Amiloride-sensitive sodium channel (ENaC) regulator of any one of claims 116 to 120, wherein the amidino compound is 6-amidino-2-napthyl 4-guanidinobenzoate.

122. The Amiloride-sensitive sodium channel (ENaC) regulator of claim 121, wherein the 6-amidino-2-napthyl 4-guanidinobenzoate is in the form of 6-amidino-2-napthyl 4-guanidinobenzoate dihydrochloride or 6-amidino-2-napthyl 4-guanidinobenzoate mesylate.

123. The Amiloride-sensitive sodium channel (ENaC) regulator of any one of claims 114 to 122, wherein increasing ciliary transport of mucus secretions is associated with the treatment of a respiratory disease.

124. The Amiloride-sensitive sodium channel (ENaC) regulator of claim 117, wherein the respiratory disease is selected from the group comprising cystic fibrosis, bronchiectasis, chronic bronchitis, chronic obstructive pulmonary disease (COPD), rhino-sinusitis and otitis media.

125. The Amiloride-sensitive sodium channel (ENaC) regulator of claim 124, wherein the respiratory disease is cystic fibrosis.

126. The Amiloride-sensitive sodium channel (ENaC) regulator of any one of claims 114 to 126, wherein the Amiloride-sensitive sodium channel (ENaC) regulator is administered in combination with a hyperosmotic agent or a purinergic agonist.

127. The Amiloride-sensitive sodium channel (ENaC) regulator of claim 126, wherein the Amiloride-sensitive sodium channel (ENaC) regulator is administered in combination with a hyperosmotic agent.

128. The Amiloride-sensitive sodium channel (ENaC) regulator of claim 127, wherein the hyperosmotic agent increases the osmolarity of the recipient's lungs to between 350 mosM to 600 mosM.

129. The Amiloride-sensitive sodium channel (ENaC) regulator of claim 128, wherein the increase in osmolarity in the recipient's lungs is achieved by inhalation as a nebulized aerosol of 0.5 ml to 20 ml of a 600 mosM to 3000 mosM solution.

130. The Amiloride-sensitive sodium channel (ENaC) regulator of claim 128, wherein the increase in osmolarity in the recipient's lungs is achieved by inhalation as a dry powder formulation of 0.5 to 3 mosmoles of a hyperosmotic agent.

131. The Amiloride-sensitive sodium channel (ENaC) regulator of claim 127, wherein the hyperosmotic agent is hypertonic saline.

132. The Amiloride-sensitive sodium channel (ENaC) regulator of claim 131, wherein the hypertonic saline increases the osmolarity of the recipient's lungs to between 350 mosM to 600 mosM.

133. The Amiloride-sensitive sodium channel (ENaC) regulator of claim 132, wherein the increase in osmolarity in the recipient's lungs is achieved by inhalation as a nebulized aerosol of 0.5 ml to 20 ml of 600 mosM to 3000 mosM hypertonic saline.

134. The Amiloride-sensitive sodium channel (ENaC) regulator of claim 127, wherein the hyperosmotic agent is mannitol.

135. The Amiloride-sensitive sodium channel (ENaC) regulator of claim 134, wherein the mannitol increases the osmolarity of the recipient's lungs to between 350 mosM to 600 mosM.

136. The Amiloride-sensitive sodium channel (ENaC) regulator of claim 135, wherein the increase in osmolarity in the recipient's lungs is achieved by inhalation as a nebulized aerosol of 0.5 ml to 20 ml of 600 mosM to 3000 mosM mannitol.

137. The Amiloride-sensitive sodium channel (ENaC) regulator of claim 135, wherein the increase in osmolarity in the recipient's lungs is achieved by inhalation as a dry powder formulation of 0.5 to 3 mosmoles of mannitol.

138. The Amiloride-sensitive sodium channel (ENaC) regulator of claim 127, wherein the hyperosmotic agent is sodium gluconate.

139. The Amiloride-sensitive sodium channel (ENaC) regulator of claim 138, wherein the sodium gluconate increases the osmolarity of the recipient's lungs to between 350 mosM to 600 mosM.

140. The Amiloride-sensitive sodium channel (ENaC) regulator of claim 139, wherein the increase in osmolarity in the recipient's lungs is achieved by inhalation as a nebulized aerosol of 0.5 ml to 20 ml of 600 mosM to 3000 mosM sodium gluconate.

141. The Amiloride-sensitive sodium channel (ENaC) regulator of claim 139, wherein the increase in osmolarity in the recipient's lungs is achieved by inhalation as a dry powder formulation of 0.5 to 3 mosmoles of sodium gluconate.

142. The Amiloride-sensitive sodium channel (ENaC) regulator of claim 126, wherein the Amiloride-sensitive sodium channel (ENaC) regulator is administered in combination with a purinergic agonist.

143. The Amiloride-sensitive sodium channel (ENaC) regulator of claim 142, wherein the purinergic agonist is uridine-5′-triphosphate (UTP), P¹,P⁴-bis(5′-uridyl) tetraphosphate tetrasodium salt (Diquafosol), or 2′-deoxycytidine(5′) tetraphospho (5′) uridine tetrasodium salt (Denufosol).

144. The Amiloride-sensitive sodium channel (ENaC) regulator of claim 142 or claim 143, wherein the purinergic agonist is administered by inhalation.

145. The Amiloride-sensitive sodium channel (ENaC) regulator of any one of claims 142-144, wherein up to 60 mg of the purinergic agonist is administered.

146. The Amiloride-sensitive sodium channel (ENaC) regulator of any one of claims 126 to 145, wherein the Amiloride-sensitive sodium channel (ENaC) regulator and hyperosmotic agent or purinergic agonist are for combined, separate or sequential administration.

147. The Amiloride-sensitive sodium channel (ENaC) regulator of any one of claims 126 to 146, wherein the Amiloride-sensitive sodium channel (ENaC) regulator and/or the hyperosmotic agent or purinergic agonist further comprises one or more pharmaceutically acceptable carriers or diluents.

148. The Amiloride-sensitive sodium channel (ENaC) regulator of any one of claims 126 to 147, wherein the Amiloride-sensitive sodium channel (ENaC) regulator and/or the hyperosmotic agent or purinergic agonist is in aqueous solution.

149. The Amiloride-sensitive sodium channel (ENaC) regulator of any one of claims 126 to 148, wherein the Amiloride-sensitive sodium channel (ENaC) regulator and/or the hyperosmotic agent or purinergic agonist also comprises one or more other active agents selected from the group of antibiotics, vaccines, decongestants (nasal or bronchial), rhDNase, non-steroidal antiinflamatory agents (NSAIDs), steroids, antiviral agents, elastase inhibitors, exofacial sodium channel blocking agents, gene therapy agents, chloride channel activators and bronchodilators.
